(19) United States
(12) Patent Application Publication
(10) Pub. No.: US 2018/0161461 A1
(43) Pub. Date: Jun. 14, 2018

(54) Rare Earth Oxide Particles and Use Thereof in Particular In Imaging

(71) Applicant: Ecole Polytechnique, Palaiseau Cedex (FR)

(72) Inventors: Markus Schoeffel, Paris (FR); Antigoni Alexandrou, Palaiseau (FR); Cedric Bouzigues, Paris (FR); Thierry Gacoin, Bures Sur Yvette (FR); Jean-Pierre Boilot, Meudon (FR)

(21) Appl. No.: 15/877,042

(22) Filed: Jan. 22, 2018

Related U.S. Application Data
(62) Division of application No. 14/383,859, filed on Sep. 8, 2014, now abandoned.

(30) Foreign Application Priority Data
Mar. 9, 2012 (FR) 1252167

(51) Int. Cl. A61K 49/18; A61K 49/00

(52) U.S. Cl. CPC A61K 49/1824; B82Y 5/00; A61K 49/0002

(57) ABSTRACT

The present application concerns multimodal composite products for imaging, in particular for diagnostic imaging, and optionally for therapy, in particular composite products which are capable of being used as contrast agents, in particular in magnetic resonance imaging (MRI), and/or in imaging techniques such as, for example, in optical imaging, in the optical detection of oxidants, in positron emission tomography (PET), in tomodensitometry (TDM) and/or in ultrasound imaging, and optionally simultaneously for use in therapy. These products are based on a particle comprising or consisting of a portion provided with a contrast agent activity and/or a paramagnetic activity, and a portion provided with a luminescent activity and optionally an oxidant detection activity.

### CROSS-REFERENCE TO RELATED APPLICATIONS

The present application is a divisional application of U.S. Ser. No. 14/383,859, which is a 35 U.S.C. §371 National Phase Application of International Patent Application No. PCT/FR2013/050,500, filed Mar. 8, 2013, claiming benefit of French Application No. 12521167, filed Mar. 9, 2012, each of which is incorporated herein by reference in its entirety.

### FIELD OF THE INVENTION

The present application relates to multimodal composite products for imaging, in particular for diagnostic imaging, and optionally for therapy, in particular composite products which are capable of being used as contrast agents, in particular in magnetic resonance imaging (MRI) and/or in imaging techniques such as, for example, in optical imaging, in the optical detection of oxidants, in positron emission tomography (PET), in tomodensitornetry (TDM) and/or in ultrasound imaging and optionally simultaneously for use in therapy. These products are based on a particle comprising or consisting of a portion provided with a contrast agent activity and/or a paramagnetic activity, and a portion provided with a luminescent activity and optionally with an oxidant detection activity.

### PRIOR ART

MRI (Magnetic resonance imaging) examinations are essentially used to image various types of soft tissues. The contrast is determined by the proton relaxation times T₁ (longitudinal relaxation) and T₂ (transverse relaxation) (Abragam, 1983 and Levitt, 2008).

The administration of a contrast agent (CA) is recommended when there is too little intrinsic contrast between the regions of interest, in clinical diagnostics generally between healthy and diseased tissues. CAs are compounds which are capable of modifying the relaxation times of water protons in the tissue in which they are present, and can thus improve a medical diagnosis in terms of superior specificity, better tissue characterization, reduction of artefacts in the image and functional information (Aime et al, 2005). Depending on the principal effect, CAs can be divided into two classes: T₁ CAs, or positive CAs, which essentially act on the longitudinal (spin-lattice) relaxation time, and T₂ CAs or negative CAs, which shorten the transverse (spin-spin) relaxation time (Bottrill et al, 2006).

The performance of a contrast agent is characterized by the relaxivity normalized to the concentration (rᵢ) (Lauffer, 1987):

$${r}_{i} = \frac{{( \frac{1}{{T}_{i}} )}_{obs} - {( \frac{1}{{T}_{i}} )}_{solv}}{[ AC ]}$$

where the first term corresponds to the inverse relaxation time of the proton in a paramagnetic CA solution, using Langevin's definition of paramagnetism, at the concentration [CA], and the second is that in the pure diamagnetic solvent, and i may be 1 or 2.
The observed relaxation rate is defined by:

$${( \frac{1}{{T}_{i}} )}_{obs} = {( \frac{1}{{T}_{i}} )}_{p} + {( \frac{1}{{T}_{i}} )}_{solv}$$

where the index p denotes the pure paramagnetic contribution of the CA.

The values are expressed in the units mM⁻¹s⁻¹ (Aime et al, 1999). The MRI signal observed for the MRI magnetic field pulse sequence types increases by increasing 1/T₁ and decreases by increasing 1/T₂, but since a CA commonly affects the two relaxation times (Caravan et al, 1999), the predominant effect in the end decides whether the CA will act as a positive CA or a negative CA.

The value of the relaxivity ratio (κ):

$$κ = \frac{{r}_{2}}{{r}_{1}}$$

can be used to determine which of the effects, T₁ or T₂, is dominant. A low value for κ of approximately 1 indicates a positive CA, while a value for the ratio κ which is substantially greater than 1 signifies that the compound acts as a negative CA.

The improvement in the proton paramagnetic relaxation of the water protons is the result of temporal fluctuations in the coupling between the magnetic moment of the electrons of the metal ion and the nuclear magnetic moment of the protons (Kowalewski et al, 1985; Banci et al, 1991; Bertini and Luchinat, 1996). At least two contributions can be distinguished: an inner sphere mechanism and an outer sphere mechanism. The inner sphere mechanism concerns molecules of solvent directly coordinated with the metallic centre, while the outer sphere relaxation refers to molecules of water in a second coordination sphere or one still further from the complex.

Depending on the actual structure of the CA, an additional contribution may be present if hydrogen bond interactions are possible with the water molecules. Since this contribution is difficult to quantify, it is often treated as an inner sphere mechanism or outer sphere mechanism depending on the strength of the hydrogen bond (Caravan et al, 1999; Aime et al, 2005).

The observed inverse relaxation time is a function of the inverse relaxation times for the two processes (Caravan et al, 1999):

$${( \frac{1}{{T}_{i}} )}_{p} = \frac{1}{{T}_{i}^{IS}} + \frac{1}{{T}_{i}^{OS}}$$

where the exponents IS and OS respectively represent the inner sphere and the outer sphere.

Currently, all clinically approved T₁ contrast agents are based on Gd³⁺, an ion having 7 unpaired electrons, chelated by organic polydentate ligands, for example with the names of “Magnevist”, “Prohance”, “Omniscan”, “OptiMark”, “Multihance”, “Eovist”, “Ablavar” and “Gadavist”, approved by the FDA (US Food and Drug Administration), and “Multihance”, “Omniscan”, “Gadovist”, “Gadograf”, “Dotarem”, “Artirem”, “Primovist”, “Gadopentetat”, “Magnegita”, “Handvist”, and “Magnetolux” approved in at least one country in the European Union.

The physical constants for some of these CAs are shown in Table 1 below (the data, with the exception of those for EOB-DTPA, are from Caravan et al, 1999 and refer here to a proton resonance frequency of 20 MHz and at the available temperature closest to 37° C. The data for EOB-DTPA for 20 MHz and 37° C. are from Vander Elst et al. (1997). The r₂ values are for the most part not reported; pK_{GdL} logarithmic dissociation constant for the Gd-ligand complexes (GdL)).

TABLE 1

| Name | Ligand | pK_{GdL} | r₁ (mM⁻¹ s⁻¹) | r₂ (mM⁻¹ s⁻¹) |
|---|---|---|---|---|
| Dotarem | DOTA | 25.3 | 3.56 | 4.75 |
| Prohance | HP-DO3A | 23.8 | 3.7 | — |
| Gadovist | DO3A-butrol | 21.1 | 4.8 | — |
| Magnevist | DTPA | 22.5 | 3.8 | — |
| Omniscan | DTPA-BMA | 16.9 | 3.96 | — |
| MultiHance | BOPTA | 22.6 | 4.39 | 5.56 |
| OptiMark | DTPA- | 16.8 | 4.7 | — |
| | BMEA | | | |
| Ablavar | MS-325 | 6.6 | — | — |
| Eovist | EOB-DTPA | — | 5.4 | — |

Furthermore, nanoparticles based on iron oxide are used as T₂ CAs. They suffer from the disadvantage of exhibiting a signal extinction effect which makes image interpretation difficult, since the resulting dark regions cannot always be attributed unambiguously to the presence of CA. In addition, the high susceptibility of the material based on iron oxide introduces magnetic field distortions into neighbouring tissues, known as susceptibility artefacts or “dazzle artefacts”, which generate darkened images and affect the background around the actual position of the agent (Butte and Kraitchrnan, 2004).

Recent advances in nanotechnology have resulted in the development of nanoparticles based on Gd³⁺ having properties of improving T₁ contrast in MRI. Nanoparticles are interesting candidates for CAs due to the increased available surface for interaction between the Gd³⁺ ions and water protons (Na et al, 2009). Nanoparticulate CAs Can be produced from an inorganic core structure carrying binding structures for paramagnetic ions (Na et al, 2009). The application of these particles results in a high local concentration of paramagnetic ions and thus in a high contrast. However, the maximum number of Gd³⁺ ions is limited by the binding sites on the surface. Another disadvantage resides in their complex synthesis involving a number of steps—at least the production of the core structure, adding the binding sites to the surface and chelating the Gd³⁺ ions into those binding sites.

These disadvantages can be overcome by using inorganic paramagnetic nanoparticles where the paramagnetic ions form an integral part of the structure of the core. In this context, the synthesis is limited to the core formation step. Many compounds containing transition metals or lanthanides would appear to be good candidates, but the majority of the research has been directed towards nanoparticles based on Gd³⁺ because of the high number of unpaired electrons in Gd³⁺ and extensive data on the properties of Gd complexes.

Thus, Hifumi et al. (2006) synthesized nanoparticles of paramagnetic GdPO₄ coated with dextran (GdPO₄/dextran) having a hydrodynamic diameter of 23 nm for the whole of the construct. In addition, Park et al. (2009) synthesized even smaller nanoparticles of Gd₂O₃ with a coating of D-glucuronic acid (GOGA). High relaxivity values compatible with a positive contrast agent were observed.

Furthermore, it is important to be able to combine the use of a particle as a contrast agent with its use for other imaging methods with complementary characteristics. In the long run, this could considerably increase the wealth of information obtained, while limiting the number of injections required to obtain this information. Further, research teams have developed instruments which are capable of simultaneously acquiring images corresponding to two different modes of imaging, in particular optical detection incorporated either into an MRI detection probe (Mastanduno et al.; 2011)or into an X ray tomograph (Alé et al; 2010). Thus, at the same time these probes can carry out oxidant detection, if it is based on optical detection.

Thus, Bridot et al. (2007) developed the preparation of nanoparticles of Gd₂O₃ with different core diameters integrated into a polysiloxane envelope (GadoSiPEG) which could also carry organic fluorophores for bimodal imaging by magnetic resonance and fluorescence.

Particles of Gd_{0.6}Eu_{0.4}VO₄ have been proposed, combining the use as MRI contrast agents, as fluorescent markers and as oxidant sensors (Schoeffel et al.; 2011). However, those particles have a low luminescence quantum yield (Q) of the order of 4%.

Furthermore, core-shell nanoparticles with a fluorescent core and a paramagnetic shell have been proposed by Zhou et al. (2011) (Nanoscale, 2011, 3, 1977). Similarly, Singh et al. (2008) (Journal of Applied Physics 104, 104307(2008)) studied core-shell nanoparticles with a luminescent core and a GdVO₄ shell. However, the authors limited themselves to doping with Eu³⁺ fixed respectively at 8% (Zhou et al, (2011)) and at 7% (Singh et al. (2008)).

Furthermore, EP 1473347 discloses core/shell nanoparticles with either a luminescent shell or a luminescent core and shell and their applications to FRET (“fluorescence resonance energy transfer”).

Thus, there is a need in the art for composite multimodal products for imaging and optionally for therapy, in particular composite products which may be used as contrast agents, in particular in MRI, and/or in other imaging techniques such as, for example, in optical imaging, in the optical detection of oxidants, in positron emission tomography (PET), in tomodensitometry (TDM) and/or in ultrasound imaging, and optionally simultaneously suitable for use in therapy.

### DESCRIPTION OF THE FIGURES

FIG. 1: Proton relaxation time in the presence of Y_{0.6}Eu_{0.4}VO₄/GdVO₄ particles as a function of the concentration of Gd³⁺. (A): T₁; (B): T₂.

FIG. 2: Luminescence emission spectrum for Y_{0.6}Eu_{0.4}VO₄/GdVO₄ particles. The luminescence was excited at 280 nm. The positions of the peaks as well as the corresponding transitions are indicated. In the case of double peaks, the position is given for each component.

FIG. 3: Detection of hydrogen peroxide with Y_{0.6}Eu_{0.4}VO₄/GdVO₄ particles. The excitation was carried out 466 nm. (A) photoreduction; laser intensity at sample: 1.6 kW/cm². Exposure time 100 ms, 1 image per s. The data were adjusted using a biexponential decay function:

$$I = {I}_{∞} + {a}_{1} exp ( - \frac{t}{{τ}_{1}} ) + {a}_{2} exp ( - \frac{t}{{τ}_{2}} )$$

(B) recovery of luminescence after adding 100 μM of hydrogen peroxide. Laser intensity at sample: 0.3 kW/cm². Exposure time 400 ms, 1 image every 3 s. The data were adjusted using a monoexponential growth function:

$$I = 1 + Δ I \cdot [ 1 - exp ( - \frac{t}{{τ}^{*}} ) ] .$$

FIG. 4: Diagrammatic representation of a particle in accordance with the invention, in section. (A) XₐL_{b}(MₚO_{q})/AₑX′_{f}(M′_{p′}O_{q′}) particle; (B) Y_{0.6}Eu_{0.4}VO₄/GdVO₄ particle.

FIG. 5: Diagrammatic representation (in section) of a coated particle in accordance with the invention. (A) XₐL_{b}(MₚO_{q})/AₑX′_{f}(M′_{p′}O_{q′}) particle covered with a third portion; (B) Y_{0.6}Eu_{0.4}VO₄/GdVO₄ particle covered with a layer of silica (SiO₂), a layer of APTES and a layer constituted by targeting molecules (“targeting”), therapeutic molecules and PEG ( [symbol] )

### DESCRIPTION OF THE INVENTION

The application provides a particle suitable for use at least both as a contrast agent, in particular for MRI, and as a luminescent agent (at least bimodal agent). This particle comprises or consists of a portion having the luminescent activity and a portion having the contrast agent activity (at least bipartite particle),

As will become apparent from the description below and the examples, the nanoparticles of the invention may thus advantageously be paramagnetic and luminescent, and in a particular embodiment, they comprise or are constituted on the one hand by a luminescent portion and on the other hand by a paramagnetic portion, the paramagnetic portion preferably being neutral in terms of luminescence. In a particular embodiment, it is the shell which is paramagnetic and neutral in terms of luminescence, i.e, it does not emit light following excitation by light or it emits with a quantum yield of less than 1%.

In a particular embodiment developed below, the particle of the invention can be used as a contrast agent, in particular in MRI, as a luminescent agent and as an oxidizing substance sensor (at least trimodal agent).

In a particular embodiment developed below, the particle of the invention is furthermore provided with a coating.

The particle of the invention comprises or consists of at least two portions, a portion with formula XₐL_{b}(MₚO_{q}), in which:
M is at least one element which is capable of associating with oxygen (O) to form an anion;
L corresponds to one or snore luminescent lanthanide ion(s);
X corresponds to one or more ion(s) which i (are) neutral in terms of luminescence: and
the values of p, q, a and b are such that the electroneutrality of XₐL_{b}(MₚO_{q}) is respected, the fraction of luminescent element, defined by the ratio b/(b+a), being from 1% to 75%; and

a portion with formula AₑX′_{f}(M′_{p′}O_{q′}), in which:
M′ is at least one element which is capable of associating with oxygen (O) to form an anion;
A corresponds to one or more paramagnetic lanthanide ions;
X′, when it is present, corresponds to one or more ion(s) which is (are) neutral in terms of paramagnetic properties; and
the values of p′, q′, e and if appropriate, f, are such that the electroneutrality of AₑX′_{f}(M′_{p′}O_{q′}) is respected, the fraction of paramagnetic element, defined by the ratio e/(e+f), being from 80% to 100%.

In a particular embodiment, the portion with formula XₐL_{b}(MₚO_{q}) is luminescent, and the portion with formula AₑX′_{f}(M′_{p′}O_{q′}) (or Aₑ(M′_{p′}O_{q′}) defined below) is paramagnetic and neutral in terms of luminescence.

In a particular embodiment the particle of the invention comprises or consists of at least two portions, a portion with formula XₐL_{b}(MₚO_{q}), in which:
M is at least one element which is capable of associating with oxygen (O) to form an anion;
L corresponds to one or more luminescent lanthanide ion(s);
X corresponds to one or more ion(s) which is (are) neutral in terms of luminescence; and
the values of p, q, a and b are such that the electroneutrality of XₐL_{b}(MₚO_{q}) is respected, the fraction of luminescent element, defined by the ratio b/(b+a), being from 1% to 75%; and
   a portion with formula AₑX′_{f}(M′_{p′}O_{q′}), in which:
M′ is at least one element which is capable of associating with oxygen (O) to form an anion;
A corresponds to one or more paramagnetic lanthanide ions;
X′ corresponds to one or more ion(s) which is (are) neutral in terms of paramagnetic properties; and
the values of p′, q′, e and f are such that the electroneutrality of AₑX′_{f}(M′_{p′}O_{q′}) is respected, the fraction of paramagnetic element, defined by the ratio e/(e+f), being from 80% to 100%.

In a particular embodiment, the particle of the invention comprises or consists of at least two portions, a portion with formula XₐL_{b}(MₚO_{q}), in which:
M is at least one element which is capable of associating with oxygen (O) to form an anion;
L corresponds to one or more luminescent lanthanide ion(s);
X corresponds to one or more ion(s) which is (are) neutral in terns of luminescence; and
the values of p, q, a and b are such that the electroneutrality of XₐL_{b}(MₚO_{q}) is respected, the fraction of luminescent element, defined by the ratio b/(b+a), being: from 1% to 75%; and
   a portion with formula Aₑ(M′_{p′}O_{q′}) in which:
M′ is at least one element which is capable of associating with oxygen (O) to form an anion:
A corresponds to one or more paramagnetic lanthanide ions; and
the values of p′, q′ and e are such that the electroneutrality of Aₑ(M′_{p′}O_{q′}) is respected.

More particularly, M, M′, L, X, p, q, a, b, A, X′ p′, q′, e and f are defined as follows:

M and M′, independently of each other, are at least one (preferably 1 or 2) element which is capable of associating with oxygen (O) to form an anion, The term “independently of each other”, means that the choice of M does not condition the choice of M′, and vice-versa. In a particular embodiment, M and M′, independently of each other, have valency +V or +VI. In a particular embodiment, M and M′ are each, independently of each other, an ion selected from the group constituted by V, P, W, Mo and As. Preferably, M and M′, independently of each other, are P or V; preferably, M and M′ are V. In one embodiment, one and/or the other of M and/or M′ represents two ions selected, independently of each other, from the group constituted by V, P, W, Mo and As. In particular, M may represent VᵥP₁₋ᵥ (v being from 0 to 1). In particular, M′ may represent V_{v′}P_{1-v′} (v′ being from 0 to 1).

L is one or more (preferably 1 or 2) luminescent lanthanide ion(s). The term “lanthanide” (or Ln) defines elements with an atomic number from 57 to 71 in the periodic classification of the elements, In one embodiment, L has a valency in the range +II and +IV, and preferably +III. In one embodiment, L is an ion selected from the group constituted by Ce, Pr, Nd, Pm, Sm, Eu, Tb, Dy, Ho, Er, Tm and Yb. In one embodiment, L is Eu, in particular Eu³⁺. In another embodiment, L is Ce, in particular Ce³⁺. In another embodiment, L is Tb, in particular Tb³⁺. In another embodiment, L represents a plurality of ions (preferably 2) selected from the group constituted by Ce, Pr, Nd, Pm, Sm, Eu, Tb, Dy, Ho, Er, Tm and Yb. In a particular embodiment, L represents the ions Ce and Tb, or the ions Er and Yb.

X corresponds to one or more (preferably 1 or 2) ion(s) which is (are) neutral in terms of luminescence. The expression “neutral in terms of luminescence», means that the ion or ions X are not capable of emitting light following excitation or emit light with a quantum yield of less than 1%, In one embodiment, X has a valency of +III. In one embodiment, X is selected from the group constituted by lanthanides and Bi. In one embodiment, X is selected from the group constituted by La, V, Gd and Bi. In one embodiment, X is selected from the group constituted by La, Y, and Bi. In a particular embodiment, X is the element yttrium (Y). In a particular embodiment, X is La. In a particular embodiment, X is as defined above and furthermore, it is not Gd.

In one embodiment, L is Eu and X is Y, such that XₐL_{b} is YₐEu_{b}. In one embodiment, L is Ce and X is La, such that XₐL_{b} is LaₐCe_{b}. In one embodiment, L is Tb and X is La, such that XₐL_{b} is LaₐTb_{b}. In one embodiment, L represents Ce and Tb, and X is La, such that XₐL_{b} is Laₐ(Ce, Tb)_{b}.

The values of p, q, a and b are such that the electroneutrality of XₐL_{b}(MₚO_{q}) is respected.

p is equal to 0 or 1, and preferably equal to 1. In one embodiment, q is in the range 2 to 5, and is preferably 4. By way of example, M is P or V, p is equal to 1, and q is equal to 4, such that (MₚO_{q}) is PO₄³⁻ or VO₄³⁻ In another example, p is equal to 0, and X is Y, such that Xₐ(MₚO_{q}) is Y₂O₃. In another embodiment, M represents the ions V and P, p is equal to 1, and q is equal to 4, such that (MₚO_{q}) is (VᵥP₁₋ᵥ)O₄.

The fraction of luminescent elements, defined by the ratio b/(b+a), is from 1% to 75%, in particular from 10% to 60% or 20% to 50%, in particular of the order of 30% or of the order of 40% (±5%). In one embodiment, the ratio b/(b+a) is less than or equal to 75%, less than or equal to 60%, less than or equal to 50%, less than or equal to 40%, less than or equal to 30% or less than or equal to 20%.

In a particular embodiment, b/b+a is more than 10%, preferably more than 20%, preferably more than 25%. In a particular embodiment, b/(b+a) is from 10% to 75% or from 20% to 75% or from 25% to 75% or from 25% to 45%.

In accordance with the invention, optimisation of the luminescence has been observed for high values of dopant ratio compared with the usual ratios. The prior art, recommends low levels of doping, of less than 10%, and choses such levels because, in bulk materials and nanoparticles, especially when they are synthesized at high temperatures, a “quenching” effect between Eu³⁺ ions appears at higher levels, which reduces the quantum yield; when these nanoparticles are excited in the UV (the absorption band of vanadate), the quantum yield for the emission of Eu³⁺ is optimal for doping values between 0.1% and 10%. In addition, when the element L is excited directly, for example Eu³⁺ ions, in the visible, as is the case according to the invention, the number of excited ions is proportional to the number of Eu³⁺ ions available in the nanoparticle. Thus, the number of photons collected, proportional to the number of excited ions multiplied by the quantum yield, is optimal for high and specific doping values. It is advantageous to have a maximum level for L without a drop in the quantum yield It has been observed that with low temperature syntheses, the quenching effect only appears at higher concentrations. For L=Eu, the optimum value for b/b+a is in the range from approximately 20% to 40%. In the case of Eu³⁺, the quantum yield is optimal for doping values in the range 20% to 40%. Since the envisaged applications of the invention are biomedical applications, excitation in the visible is preferable to excitation in the UV, which is more damaging to cells and absorbed more by the tissues.

In one embodiment, L is Eu and the ratio b/(b+a) is 40%, such that XₐL_{b} is X_{0.6}Eu_{0.4}. In one embodiment, L is Eu, X is Y and the ratio b/(b+a) is 40%, such that XₐL_{b} is Y_{0.6}Eu_{0.4}. a and b are such that a+b=1.

In one embodiment, X is Y, L is Eu, M is V or P, and the ratio b/(b+a) is from 1% to 75%, preferably 10% to 75%, still more preferably 20% to 75%. In one embodiment, X is Y, L is Eu, M is V and the ratio b/(b+a) is from 1% to 75%, preferably 10% to 75%, still more preferably 20% to 75%. In one embodiment, X is Y, L is Eu, M is V and the ratio b/(b+a) is 40%, such that XₐL_{b}(MₚO_{q}) is Y_{0.6}Eu_{0.4}(VO₄).

In one embodiment, Lis Eu and X is Y, M is V and/or P, and the ratio b/(b+a) is from 1% to 75%, preferably 10% to 75%, still more preferably 20% to 75%. In one embodiment, L is Ce and X is La, M is V and/or P, and the ratio b/(b+a) is from 1% to 75%, preferably 10% to 75%, still more preferably 20% to 75%. In one embodiment, L represents Ce and Tb, and X is La, M is V and/or P, and the ratio b/(b+a) is from 1% to 75%, preferably 10% to 75%, still more preferably 20% to 75%.

A represents one or more (preferably 1 or 2) paramagnetic ion(s) from the lanthanides family. The term “paramagnetic” as used herein has its normal meaning, more particularly in accordance with Langevin's definition of paramagnetism. In one embodiment, A is a paramagnetic ion selected from the group constituted by Ce, Pr, Nd, Eu, Gd, Tb, Ho, Er, Tm and Yb. In a particular embodiment, A is Gd. In one embodiment, L and A are different. In another embodiment, A represents several paramagnetic ions (preferably 2) selected from the group constituted by Ce, Pr, Nd, Eu, Gd, Tb, Ho, Er, Tm and Yb. In one embodiment, A represents the ions Gd and Eu. In one embodiment, A is different from L, in the number and/or nature of the ions.

A particular advantage according to the invention lies in the choice of a major element in the matrix which is not luminescent in the matrix, the paramagnetic shell possibly being neutral in terms of luminescence. Thus, for example, for one of the paramagnetic elements used in the invention in the form of Gd³⁺, GdVO₄ and GdPO₄ but also Gd₂O₃ and other salts and oxides of Gd³⁺ are neutral in terms of luminescence in the form of nanoparticles, or of shells in core-shell systems. In one embodiment, the elements of the shell are neutral in terms of luminescence.

X′, when it is present, corresponds to one or more ion(s) (preferably 1 or 2) which are neutral in terms of paramagnetic properties. The expression “neutral in terms of paramagnetic properties” means that the ion or ions X does/do not have unpaired electron spins in the ground state. The “neutrality in terms of paramagnetic properties” of the ion(s) X′ as used herein has its usual meaning, more particularly in accordance with Langevin's definition of paramagnetism. In one embodiment, X′ has a valency of +III. In one embodiment, X′ is selected from the group constituted by lanthanides and Bi. In one embodiment, X′ is selected from the group constituted by La, Y and Bi. In a particular embodiment, X′ is the element Yttrium (Y).

The values of p′, q′, e and, if appropriate, f are such that the electroneutrality of AₑX′_{f}(M′_{p′}O_{q′}) is respected.

p′ is equal to 0 or 1, and preferably equal to 1. In one embodiment, q′ is in the range 2 to 5, and is preferably 4. By way of example, M′ is P or V, p′ is equal to 1 and q′ is equal to 4, such that (M′_{p′}O_{q′}) is PO₄³⁻ or VO₄³⁻. In another embodiment, M′ represents the ions V and P, p′ is equal to 1, and q is equal to 4, such that (M_{p′}O_{q′}) is (V_{v′}P_{1-v′})O₄.

The fraction of paramagnetic element, defined by the ratio e/(e+f), is from 80% to 100%, in particular from 90% to 100% or from 95% to 100%. In one embodiment, the ratio e/(e+f) is greater than or equal to 80%, 90% or 95%. In one embodiment, the ratio e/(e+f) is greater than or equal to 80%, 90% or 95%, and less than 100%. In one embodiment, M is V or P, A is Gd and the ratio e/(e+f) is from 80% to 100%. In one embodiment, M is V, A is Gd and the ratio et(e+f) is from 80 to 100%. e and f are such that e+f=1.

In one embodiment, the ratio e/(e+f) is 100%, i.e. f is equal to 0, such that AₑX′_{f}(M′_{p′}O_{q′}) is Aₑ(M′_{p′}O_{q′}), the values of p′, q′ and e being such that the electroneutrality of Aₑ(M′_{p′}O_{q′}) is respected. In one embodiment, A is Gd and the ratio e/(e+f) is 100%, such that AₑX′_{f}(M′_{p′}O_{q′}) is Gd(M′_{p′}O_{q′}). In one embodiment, M is V, A is Gd and the ratio e/(e+f) is 100%, such that AₑX′_{f}(M′_{p′}O_{q′}) is Gd(VO₄).

The particle of the invention may also be defined as comprising or consisting of two portions:
a portion with formula XₐL_{b}(MₚO_{q}), in which M, L, X, p, q, a and b are as defined above and selected such that the portion XₐL_{b}(MₚO_{q}) has luminescent activity; and
a portion with formula AₑX′_{f}(M′_{p′}O_{q′}) in which M′, A, X′ when it is present, p′, q′, e and if appropriate, f, are as defined above, and selected such that the portion AₑX′_{f}(M′_{p′}O_{q′}) has a contrast agent activity, in particular in MRI and/or a paramagnetic activity.

The term “provided with luminescent activity” or “can be used as a luminescent agent”, means a particle (or composition comprising particles) which is capable of emitting light following excitation. The luminescent activity of a particle may be determined by calculating the luminescence quantum yield (Q), which corresponds to the ratio between the number of photons emitted and the number of photons absorbed (the higher Q is, the more luminescent is the particle). A particle (in its uncoated form) will be considered to be an effective luminescence agent when the value of Q is 10% or more, preferably at least 20% (see Example 1.7).

The term “provided with contrast agent activity» or “can be used as a contrast agent” means a particle (or composition comprising particles) which reduces the relaxation times T₁ and/or T₂ when used in MRI. The contrast agent activity of a particle may be assessed by determining on the one hand the relaxivities r₁ and r₂, and on the other hand by determining the relaxivity ratio, r₂/r₁=κ. The values r₁ and r₂ are defined by the slopes of the straight lines for the relaxation rates 1/T₁, and 1/T₂, respectively, as a function of the concentration of the particles (see Examples 1.5 and 1.6).

Preferably, the particles of the invention can be used as a T₁ contrast agent, i.e. exhibit a preponderantly T₁ effect. In this embodiment, a particle is considered to be an effective T₁ contrast agent when the values of r₁ and r₂ are at least approximately 4 mM⁻¹s⁻¹, and the ratio r₂/r₁ (κ) is of the order of 1, preferably in the range 1 to 2, in particular 1 to 1.5.

The application in particular envisages a particle in accordance with the invention which can be used as a contrast agent, in particular in MRI, as a luminescent agent and as an oxidizing substance sensor (at least trimodal agent). Thus, the particle of the invention comprises or consists of two portions, a portion provided with luminescent activity and oxidizing substance detection activity, and another portion provided with contrast agent activity.

In this embodiment, the particle is defined as comprising or consisting of two portions:
a portion with formula XₐL_{b}(MₚO_{q}) in which M is V, L is Eu, and X, a, b and p are as defined above and selected such that the portion XₐEu_{b}(VₚO_{q}) has a luminescent activity and an oxidizing substance detection activity; and
a portion with formula AₑX′_{f}(M′_{p′}O_{q′}), in which M′, A, X′ when it is present, p′, q′, e and, if appropriate, f are as defined above and selected such that the portion AₑX′_{f}(M′_{p′}O_{q′}) has a contrast agent activity, in particular in MRI.

Thus, the particle of the invention comprises or consists of at least two portions, with a portion being with formula XₐEu_{b}(VₚO_{q}) and a portion being with formula AₑX′_{f}(M′_{p′}O_{q′}), in which:
X corresponds to one or more, preferably one or two, ion(s) which is (are) neutral in terms of luminescence;
the values of p, q, a and b are such that the electroneutrality of XₐEu_{b}(VₚO_{q}) is respected, the fraction of luminescent element, defined by the ratio b/(b+a), being from 1% to 75%; and
M′ is at least one element which is capable of associating with oxygen (O) to form an anion;
A corresponds to one or more, preferably one or two, paramagnetic lanthanide ion(s);
X′, when it is present, corresponds to one or more ions which are neutral in terms of paramagnetic properties; and
the values of p′, q′, e and, if appropriate, f are such that the electroneutrality of AₑX′_{f}(M′_{p′}O_{q′}) is respected, the fraction of paramagnetic element, defined by the ratio e/(e+f), being from 80% to 100%.

The term provided with an activity “as a sensor agent for oxidizing substances” or “an activity of detection of oxidizing substances”, means a particle (or composition comprising particles) which is capable of detecting, quantitatively, the concentration of oxidizing substances (such as hydrogen peroxide, H₂O₂, the hypochlorite anion), intracellularly or in vivo. In a particular embodiment, the detection of the concentration of oxidizing substances is dynamic, i.e. it is possible to detect the concentration as a function of time. In another embodiment, the particles of the invention are used as a sensor agent for hydrogen peroxide.

A particle will be considered as an effective sensor for oxidizing substances, in particular hydrogen peroxide, when the luminescent ions can be oxidized reversibly by the oxidizing substances, producing a modulation in their intensity of luminescence in a given wavelength band. In one embodiment, the luminescent ions are photoreduced by irradiation before they are used for the detection of oxidizing substances (Casanova et al.; 2009), In this case, the photoreduction induces a reduction in the luminescence of the luminescent ion which is at least 10%, preferably greater than or equal to 20%, greater than or equal to 30%, greater than or equal to 40% or greater than or equal to 50%. In another embodiment, the luminescent ions are already in a valency state such that they are capable of undergoing oxidation. The modulation in the intensity of the luminescence produced by concentrations of oxidants at physiological and pathophysiological concentrations must be sufficiently high, above the noise, to be able to be detected (see Example 1.8). In this case, the ratio between the luminescence recovery signal and the noise is more than 1, preferably more than 2 or preferably more than 5. In one particular embodiment, in combination or independently of the preceding embodiment, the characteristic time necessary to obtain this recovery is of the order of a minute, preferably less than 5 min, preferably less than 1 min or preferably less than 30 s.

The particle of the invention has a formula XₐL_{b}(MₚO_{q})/AₑX′_{f}(M′_{p′}O_{q′}) or a formula XₐL_{b}(MₚO_{q})/Aₑ(M′_{p′}O_{q′}), in particular a formula XₐEu_{b}(VₚO_{q})/AₑX′_{f}(M′_{p′}O_{q′}) or a formula XₐEu_{b}(VₚO_{q})/Aₑ(M′_{p′}O_{q′}). In particular, the particle of the invention has a formula XₐEu_{b}(VO₄)/AₑX′_{f}(M′_{p′}O_{q′}) or a formula XₐEu_{b}(VO₄)/Aₑ(M′_{p′}O_{q′}).

In a particular embodiment, the particle of the invention has a formula selected from the group constituted by YₐEu_{b}(VO₄)/Gd(VO₄), YₐEu_{b}(PO₄)/Gd(VO₄), YₐEu_{b}(VO₄)/Gd(PO₄) and YₐEu_{b}(PO₄)/Gd(PO₄), the ratio b/(b+a), being from 1% to 75%, in particular from 10% to 60% or 20% to 50%, in particular of the order of 30% or of the order of 40% (±5%). In one embodiment, the particle of the invention has the formula Y_{0.6}Eu_{0.4}(VO₄)/Gd(VO₄).

In one embodiment, at least one (preferably only 1) of M, M′, L, X, A and, if appropriate, X′ is in the form of a radio-isotope. In a particular embodiment, L is in the form of a radio-isotope, for example ⁸⁶Y. In one embodiment, the surface of the nanoparticles is functionalized with organic chelating agents, for example with the ligand DOTA, in order to allow binding of a radio-isotope appropriate to the emission of positrons, such as ⁶⁴Cu or ⁸⁶Y. In another embodiment, the functionalization of the surface is carried out with organic molecules containing, for example, the ions ¹¹C, ¹³N, ¹⁸F, also appropriate for the emission of positrons.

In the context of the present application, the term “portion” means a structure with a formula as indicated above, irrespective of its spatial arrangement with the other portion, excluding a homogeneous mixture of the two portions. For this reason, the particles are defined as composites.

Thus, in one embodiment, the at least two portions with formula XₐL_{b}(MₚO_{q}) and AₑX′_{f}(M′_{p′}O_{q′}) or with formula XₐL_{b}(MₚO_{q}) and Aₑ(M′_{p′}O_{q′}), respectively constituting the luminescent portion and the paramagnetic portion of the particle of the invention, are juxtaposed, i.e. they are in contact with each other without the two portions being mixed together or in a manner such that only a small proportion of the whole is present as a mixture (less than 10% for each of the portions). Thus, one of the phases may be at least partially dispersed in the other.

In another embodiment, the at least two portions with formula XₐL_{b}(MₚO_{q}) and AₑX′_{f}(M′_{p′}O_{q′}) or with formula XₐL_{b}(MₚO_{q}) and Aₑ(M′_{p′}O_{q′}) constituting at least one zone of the particle of the invention are arranged in a gradient structure, such that at least one zone of the particle is constituted by 100% of one portion, another zone is constituted by 100% of the other portion, and that between these two zones the two portions are mixed such that the proportion of one of the portions decreases while the proportion of the other portion increases in accordance with a gradient.

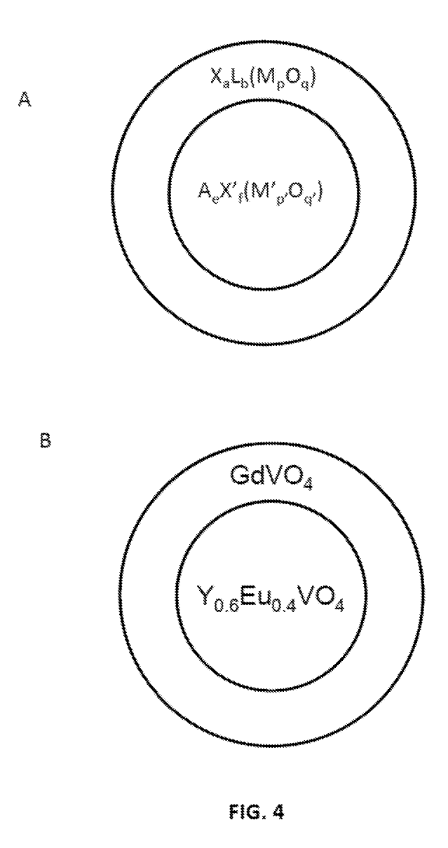

In another embodiment, the at least two portions with formula XₐL_{b}(MₚO_{q}) and AₑX′_{f}(M′_{p′}O_{q′}) or with formula XₐL_{b}(MₚO_{q}) and Aₑ(M′_{p′}O_{q′}) constituting the particle of the invention are arranged in a so-called core/shell structure, generally spherical or spheroidal, in which one of the portions is in the centre of the particle and forms the core, completely surrounded by the other portion, termed the shell (FIG. 4A).

In one embodiment of this core/shell structure, the portion forming the core is not mixed with the shell. In another embodiment of this core/shell structure, at the limit of the core and of the shell is an intermediate zone where a small portion (less than 10% for each of the portions) of each of the two portions is mixed with the other.

In a particular embodiment, whether or not the two portions are mixed, the portion with formula XₐL_{b}(MₚO_{q}) [in particular the portion with formula XₐEu_{b}(VₚO_{q})] constitutes the core of the particle, and the portion with formula AₑX′_{f}(M′_{p′}O_{q′}) or with formula Aₑ(M′_{p′}O_{q′}) constitutes the shell of the particle. Thus, for a particle with formula Y_{0.6}Eu_{0.4}(VO₄)/Gd(VO₄), the portion Y_{0.6}Eu_{0.4}(VO₄) constitutes the luminescent core of the particle, and the portion Gd(VO₄) constitutes the paramagnetic shell of the particle (FIG. 4B). Nanoparticles comprising a portion YₐEu_{b}(P,V)O₄ and a portion Gd(P,V)O₄ and in which b/b+a is more than 10% and may be up to 75% or is from 20% to 75% or from 25% to 75% or from 25% to 45% are particularly preferred, and more particularly these nanoparticles YₐEu_{b}(V,P)O₄/Gd(V,P)O₄, where the portion with formula YₐEu_{b}(P,V)O₄ constitutes the core of the particle, and the portion with formula Gd(VO₄) constitutes the shell thereof.

Other preferred embodiments are also the nanoparticles La₁₋ₓEuₓPO₄/GdPO₄, the nanoparticles La₁₋ₓEuₓP_{y}V_{1-y}O₄/GdPO₄ and the nanoparticles Y₁₋ₓEuₓP_{y}V_{1-y}O₄/GdVO₄ with x being from 10% to 75% and y being from 0.1% to 99%.

In a particular, advantageous embodiment, the core is neutral in terms of paramagnetism and/or the shell is neutral in terms of luminescence.

In a particular embodiment of the particles of the invention, the volumetric fraction of the shell (% vol), i.e. the volume of the shell with respect to the total volume of the nanoparticle, is in the range 5% to 95%, preferably in the range 25% to 75%, preferably in the range 50% to 60%. In a particular embodiment, the volumetric fraction of the shell does not exceed 60%. In a particular embodiment, the volumetric fraction of the shell is of the order of 58±5% of the total volume of the nanoparticle.

In a particular embodiment, the volume fraction of the core [with respect to the particle as a whole] may be in the range 5% to 95%, preferably in the range 25% to 75%, preferably in the range 40% to 50%. In a particular embodiment, the volume fraction of the core does not exceed 50%.

The application also proposes a composition comprising particles with formula XₐL_{b}(MₚO_{q})/AₑX′_{f}(M′_{p′}O_{q′}) or with formula XₐL_{b}(MₚO_{q})/Aₑ(M′_{p′}O_{q′}). In a particular embodiment, the particles have the same composition, i.e. the nature of X, L, M, X′ when it is present, A and M′ and the value of p, q, p′, q′, e and f are identical for all of the particles of the composition, the value of a and b possibly varying. In another embodiment, the particles have the same composition and the same nature, i.e. the nature of X, L, M, X′ when it is present, A and M′ and the value of p, q, p′, q′, a, b, e and f are identical for all of the particles of the composition.

In another embodiment, the composition comprises different particles of the invention which may vary in the nature of X, L, M, X′ when it is present, A and/or M′, and/or in the value of a, b, p, q, p′, q′, e and/or f. In one embodiment, the particles of the invention differ solely in the nature of X and X′ when it is present, and optionally in the values of a and b. In another particular embodiment, the particles of the invention contained in the composition differ solely in the nature of L, and optionally in the values of a and b. In another embodiment, the particles of the invention differ solely in the nature of X, and optionally in the values of a and b.

The portions of the particle of the invention may contain one or more crystalline zone(s) of the metal oxide(s). In a particular embodiment, the structure of one and/or the other of the portions of the particle is not monocrystalline. If several crystalline domains are present within the particle, these domains are preferably crystals with the same direction. However, within a composition of particles of the invention, certain particles may possibly have amorphous structural domains. Thus, in a composition of particles in accordance with the invention, more than 50%, more than 70%, more than 80% or more than 90%, more than 95%, more than 98%, more than 99% or 100% of the particles have a crystalline structure. In addition, domains with an amorphous structure may possibly exist within the particles of the invention. In a particular embodiment, more than 50%, more than 70%, more than 80%, more than 90%, more than 95%, more than 98%, more than 99% or 100% of the volume of the particle has a crystalline structure.

The particles of the invention may be porous or non-porous, i.e. the particles have or respectively have not the capacity to allow water in particular to penetrate into the particle. In a particular embodiment, the particles of the invention are porous. Furthermore, in the context of a composition of particles in accordance with the invention, more than 50%, more than 70%, more than 80%, more than 90%, more than 95%, more than 98%, more than 99% or 100% of the particles are porous. In addition, within the particles of the invention, a fraction of the volume of each particle may be porous. Thus, in the particles in accordance with the invention, more than 20%, more than 50%, more than 70%, more than 80%, more than 90%, more than 95%, more than 98%, more than 99% or 100% of the volume of the particle is porous.

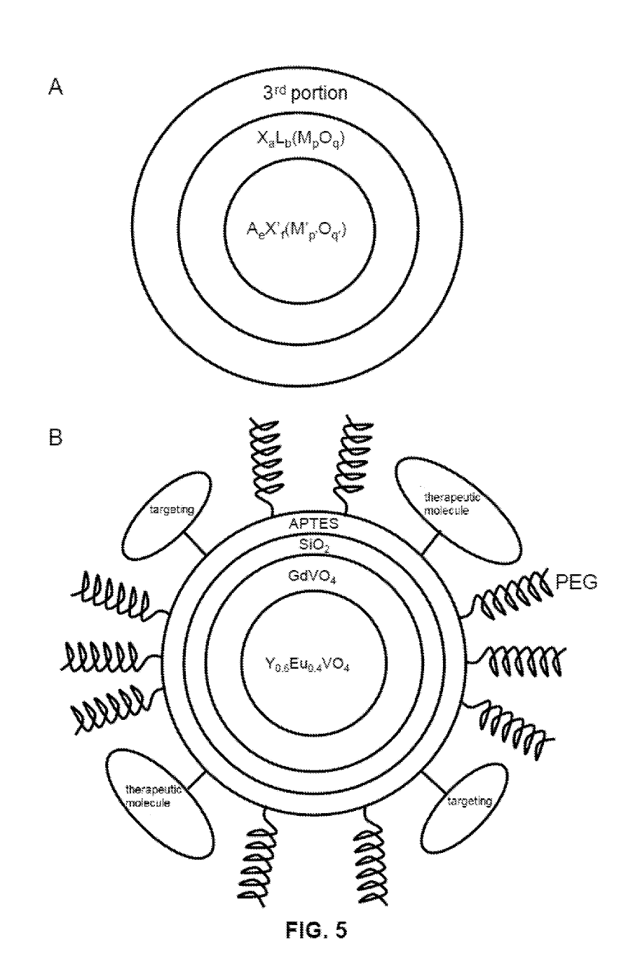

In a particular embodiment, the invention also proposes a particle of the invention constituted by the two portions XₐL_{b}(MₚO_{q}) and AₑX′_{f}(M′_{p′}O_{q′}) or constituted by the two portions XₐL_{b}(MₚO_{q}) and Aₑ(M′_{p′}O_{q′}) as defined herein (constituting the particle in its uncoated form), further provided with a third portion in order to produce a coated particle. The third portion surrounds the uncoated particle. In a particular embodiment, the coated particle consists of a core surrounded by a shell, itself surrounded by this third portion (FIG. 5A).

This third portion comprises at least one, preferably one, two or three, layer(s) selected from a preparation layer, a layer carrying functional groups and a layer constituted by biologically active molecules; the layers being as defined below.

Thus, in a particular embodiment, this third portion consists of a preparation layer, such that the uncoated particle of the invention is coated only with a preparation layer.

In another embodiment, this third portion consists of a preparation layer and a layer carrying functional groups, such that the uncoated particle of the invention is covered with a preparation layer and a layer carrying functional groups (functionalized particles). In a particular embodiment, the preparation layer is innermost with respect to the layer carrying the functional groups, i.e. the preparation layer is applied to the uncoated particle and the layer carrying the functional groups is applied over the preparation layer.

In another embodiment, this third portion consists of a preparation layer and a layer constituted by biologically active molecules, such that the uncoated particle of the invention is coated solely by a preparation layer and by a layer constituted by biologically active molecules. In a particular embodiment, the preparation layer is innermost with respect to the layer constituted by biologically active molecules, i.e. the preparation layer is applied over the uncoated particle, and the layer constituted by biologically active molecules is applied over the preparation layer.

In another embodiment, this third portion consists of a preparation layer, a layer carrying functional groups and a layer constituted by biologically active molecules, such that the uncoated particle of the invention is coated with a preparation layer, a layer carrying functional groups and a layer constituted by biologically active molecules. In a particular embodiment, the preparation layer is innermost with respect to the layer carrying functional groups, and the layer carrying functional groups is itself innermost with respect to the layer constituted by biologically active molecules, i.e. the preparation layer is applied over the uncoated particle, the layer carrying functional groups is applied over the preparation layer, and the layer constituted by biologically active molecules is applied over the layer carrying functional groups.

In a particular embodiment, this third portion has neither its own contrast agent activity, nor its own luminescent activity, and if appropriate, nor is own oxidizing substances detection activity. In a particular embodiment, this third portion has neither contrast agent activity and, if appropriate, nor does it have oxidizing substance detection activity, but has a luminescent activity which is distinct from that of the luminescent ions (L) contained in the particle of the invention. In a particular embodiment, this distinct luminescence activity is exerted by molecules (in particular fluorophores) contained in one of the three layers of the coating, preferably in the preparation layer or in the layer carrying functional groups. This distinct luminescence activity is distinguished from the luminescence activity of the luminescent ions (L) contained in the particles by its colour, its photophysical properties and/or its sensitivity to environmental factors, such as pH or the concentration of ions such as Ca²⁺.

The term “biologically active molecule”, means any molecule of natural or synthetic origin such as chemical compounds, proteins, polypeptides or polynucleotides, which is or are selected as a function of the desired activity.

In a particular embodiment, the biologically active molecule or molecules is or are targeting molecules, i.e. molecules which will allow the specific targeting of the particle of the invention towards an organ, a body fluid (for example blood), a cell type (for example platelets, lymphocytes, monocytes, tumour cells, etc.) or a cell compartment. Thus, this specific targeting may be accomplished with the aid of an antibody, monoclonal or polyclonal, or protein or polypeptide ligands of cell receptors. Non-limiting examples which may be cited are the following receptor/ligand pairs: TGF/TGFR, EGF/EGFR, TNFα/TNFR, interferon/interferon receptor, interleukin/interleukin receptor, GMCSF/GMCSF receptor, MSCF/MSCF receptor, and GCSF/GCSF receptor. Other ligands which may be cited are toxins or detoxified toxins and their cell receptors. Regarding antibodies, they will be selected as a function of the antigen or antigens against which the antibody/antibodies is or are directed. In a particular embodiment, it is possible to use antibodies recognizing antigens located on monocytes, lymphocytes or platelets, for example the antibodies marketed by Santa Cruz Biotechnology (http://www.scbt.com/).

In another embodiment, the biologically active molecule or molecules are fluorescent molecules, and for example are in the form of fusion proteins with fluorescent proteins.

In another embodiment, the biologically active molecule or molecules are stealth agents, such as polyethylene glycol (PEG) or dextran, so that the particles are rendered stealthy to the organism and thus their circulation time in the blood can be increased.

In another embodiment, the biologically active molecule or molecules are molecules with a therapeutic activity, in particular anticancer molecules (chemotherapeutic). Examples of chemotherapeutic molecules are: Cisplatin, Methotrexat, Bleomycin, Cyclophosphamid, Mitomycin, 5-Fluoruracil, Doxorubicin/Adriamycin and Docetaxel. The use of the particle of the invention as a vehicle for transporting therapeutic molecules (drugs) has a number of advantages: the particles encapsulating the drugs generally have a longer circulation time in the body than molecular drugs, and the particles can eliminate the multiple resistance effect to drugs of tumour cells, where molecular drugs are readily eliminated from the cell by pumping via the membrane pumps (Kim et at, 2009).

In another embodiment, the particle in accordance with the invention carries at least two, preferably two or three, types of biologically active molecules on its surface, selected from those described above. In a particular embodiment, the particle carries targeting molecules and stealth molecules as defined above, In another embodiment, the particle carries targeting molecules and therapeutic molecules as defined above, and optionally stealth molecules as defined above. Thus, the particles of the invention in accordance with this latter embodiment can be used to avoid unwanted secondary effects linked to the transport of therapeutic molecules in a non-pathogenic tissue.

Irrespective of the embodiment, the biologically active molecules may be attached to the surface of the particle or, if appropriate, to the preparation layer, directly or via a layer carrying functional groups, by covalent or non-covalent bonding. Attachment of these biologically active molecules is carried out using conventional techniques of oxidation, halogenation, alkylation, acylation, addition, substitution or amidation of the surface of the particle, the preparation layer and/or the layer carrying the functional groups, with the biologically active molecules.

The preparation layer is applied directly to the particle, either by covalent bonding or by adsorption. This preparation layer may be hydrophilic or hydrophobic. In a particular embodiment, this preparation layer is amorphous.

In one embodiment, the preparation layer is constituted by molecules, non-covalently bound to the particle, of which the charge is opposite to that of the uncoated particle of the invention. Examples of such binding molecules are anionic, cationic or zwitterionic detergents, peptides, acidic or basic proteins, polyamines, polyamides as well as polysulphonic or polycarboxylic acids. These binding molecules may be adsorbed onto the surface of the particle by co-incubation.

In a particular embodiment, the preparation layer is constituted by silica (SiO₂) (silica particles). By way of example, the layer of silica may be formed by condensation of an appropriate precursor containing the silicon atom around the particle of the invention. In this case, the layer of silica is bound to the particle of the invention by means of electrostatic forces. In one particular embodiment, the layer of silica is formed from sodium metasilicate (Na₂SiO₃) in accordance with the following reaction (where “RE” represents A and/or X′ in the context of a particle in accordance with the invention):

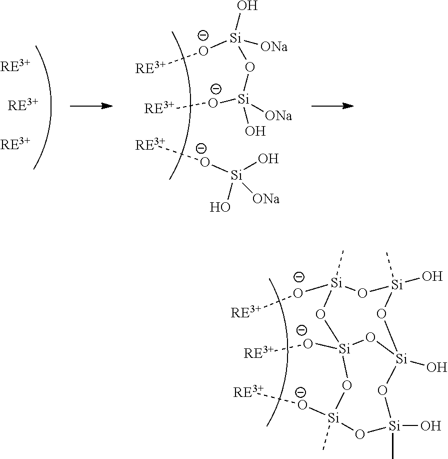

The layer carrying the functional groups, when it is present, provides the bond between the preparation layer on the one hand and the layer carrying the biologically active molecules on the other hand. It is constituted by organic groups, for example organosilanes carrying amine, thiol or carboxyl functions. A particle carrying a preparation layer and a layer with functional groups as described herein is termed a functionalized particle. In one embodiment, the layer carrying the functional groups is formed from (3-aminopropyl) triethoxysilane (APTES), which carries amine groups. As an example, the amine groups are added to the particle of the invention, as a first step, by hydrolysis of the ethoxy groups of the APTES in order to generate hydroxyl groups which, in a second step, can condense with the hydroxyl groups of the preparation layer in order to form a covalent bond in accordance with the reaction scheme below (where ‘RE’ represents A and/or X′ in the context of a particle in accordance with the invention):

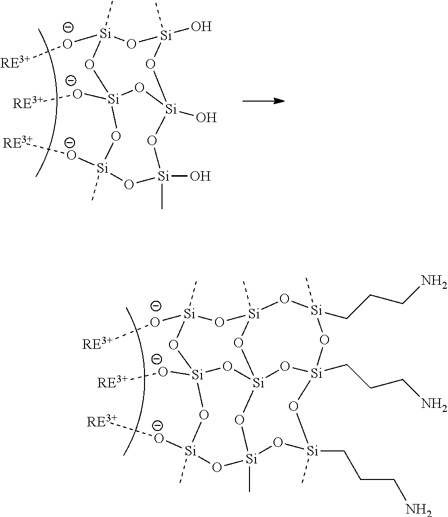

The particle which is functionalized in this manner may be bound to biologically active molecules (to form the layer constituted by biologically active molecules) by any means known to the skilled person, for example a weak chemical bond such as, for example, an electrostatic force, a Van der Waals force, hydrogen bond, hydrophobic bonds or by a strong chemical bond such as, for example, an ionic, covalent or metallic bond, or by means of a coupling agent such as, for example, coupling agents carrying double functions which can be used to attach them on the one hand to the functions (for example amine functions or carboxylic acid functions) present at the surface of the particle, and on the other hand to functions of the targeting molecule (for example amine functions or sulphhydryl functions). The functionalized particle and the biologically active molecule or molecules may also be bound using, for example, biological interactions with a strong affinity such as the biotin-streptavidin interaction (or ligand-receptor interaction or antibody-antigen interaction), and a multiple step coupling, i.e. initial coupling of streptavidin (or biotin) to the functionalized particle and coupling the biotin (or streptavidin) to the biologically active molecule or molecules, and then interaction of the two coupling products. It is also possible to cite coupling techniques between, for example, a carboxyl group and a carbodiimide, between an amine and an N-hydroxysuccinimide or an imidoester and between a thiol and a maleimide.

When the functionalized particle carries amine groups (such as, for example, after treatment with (3-aminopropyl) triethoxysilane), in order to couple the biologically active molecule or molecules, it is possible to use binding agents such as (1) bis(sulphosuccinimidyl)suberate (BS₃), a homobifonctional binding agent which, by means of its N-ester hydroxysulphosuccinimide (NHS) groups, forms bonds with the amine groups carried by various molecules, (2) 1-ethyl-3-(3-dimethylaminopropyl) carbodiimide (EDC), a carbodiimide binding agent which activates the carboxyl groups for a spontaneous reaction with primary amines, and (3) sulphosuccinimidyl-4-(N-maleimidomethyl)cyclohexane-1-carboxylate (Sulpho-SMCC) which, by means of its sulpho-NHS ester group, binds molecules containing a primary amine and which, by means of its maleimide group, reacts with cysteine residues.

By way of example, the functionalized particle of the invention may be coupled to a protein or a polypeptide having amine functions at its surface via bis(sulphosuccinimidyl)suberate (BS₃). The coupling method, described in detail by Casanova et al (2007), comprises:)
i) optionally, selecting the particles of the invention by size by means of centrifugation;
ii) transferring the particles from the aqueous solvent to a dimethylsulphoxide (DMSO) solvent;
iii) a first reaction of acylation between the particles and the cross-linker BS₃;
iv) transferring the particles from DMSO to an aqueous solvent and a second reaction between the particles/BS₃ complex and the protein or the polypeptide to be coupled; and
v) separation by centrifugation, of the free proteins or polypeptides from the particles coupled to the proteins or polypeptides.

The ratio of the concentrations of the particles of the invention and the proteins or polypeptides is selected as a function of the number of proteins or polypeptides which are to be coupled per particle. When a single molecule is to be fixed to the particle and when the reaction of step iv) has an efficiency of close to 100%, a ratio of the concentrations of particles and proteins of interest which is close to 1:1 obtained when carrying out step iv). The concentration of particles coupled to BS₃ and the proteins or polypeptides before carrying out step iv) can be determined by their absorption. After carrying out this step iv), since the absorption of the proteins or polypeptides and the particles are superimposed, the concentration of the proteins or polypeptides can be determined using standard tests such as the Bradford test.

In a second example, the functionalized particle of the invention may be coupled to aminated PEG, in particular to turn them into stealth particles. Steps i) to v) described above are repeated in an identical manner, with PEG replacing the protein or the polypeptide to be coupled from step iv). In step iv), a ratio of PEG/particle concentrations of 10:1, 20:1 or 40:1 is used in order to provide complete coverage of the surface of the particle with PEG. It is also possible to couple both PEG and a protein or a polypeptide to the particles by selecting a particles/proteins/PEG ratio of concentrations which is suitable. Thus, in step iv) described above, the second reaction will, for example, take place between a concentration C of particles, a concentration 2 C of proteins and 10 C of PEG.

Irrespective of the embodiment of the invention, in its uncoated, coated or functionalized form, the particle may have a spheroidal shape (including a spherical particle) or any other irregular shape.

The dimensions of the particles of the invention (defined as the diameter for spherical particles and as the largest dimension when the particle is spheroidal in shape), is in the range 1 to 500 nm. In particular, in its uncoated form, the particle size is less than 200 nm, in particular less than 100, less than 50, less than 25 or less than 10 nm. In an embodiment in which the particle is coated or functionalized, the dimensions will be greater than that of a uncoated particle and less than 200 nm, in particular less than 100 nm, less than 50 nm or less than 25 nm. The particles can be defined as nanoparticles (NP).

In the context of a composition of the invention, the particle dimensions may be uniform (or monodispersed), i.e. more than 75%, in particular more than 80% or more than 90% of the particles have a dimension which differs from the average dimension of all of the particles of said composition by at most 50 nm, at most 40 nm, at most 30 nm, at most 20 nm or at most 10 nm. In another embodiment of a composition which is uniform in dimensions, the size distribution of more than 75%, in particular more than 80% or more than 90% of the particles is in the range of sizes which is ±40%, ±30%, ±20% or ±10% of the mean particle size. Particles in a composition with dimensions which do not satisfy one of the two dimensions above are known as polydispersed.

The present application also proposes a method for the preparation of particles in accordance with the invention, which comprises or consists of:
(1) synthesizing portions with formula XₐL_{b}(MₚO_{q}), by a coprecipitation reaction between an aqueous solution containing the elements X and L with an aqueous solution containing an oxo-hydroxo salt of the element M;
(2) coating the portions with formula XₐL_{b}(MₚO_{q}) synthesized in (1) with a portion with formula AₑX′_{f}(M′_{p′}O_{q′}) or with a portion with formula Aₑ(M′_{p′}O_{q′}), by a coprecipitation reaction, in the presence of portions with formula XₐL_{b}(MₚO_{q}) synthesized in (1), between an aqueous solution containing the elements X′ and A or containing the element A (when X′ is absent) with an aqueous solution containing an oxo-hydroxo salt of the element M′; and
(3) recovering the particles with formula XₐL_{b}(MₚO_{q})/AₑX′_{f}(M′_{p′}O_{q′}) or with formula XₐL_{b}(MₚO_{q})/Aₑ(M′_{p′}O_{q′}).

In a particular embodiment, the aqueous solution containing the elements X and L is in the form of chlorides, de nitrates or acetates. In a particular embodiment, the aqueous solution containing the elements X and L may also contain complexing agents for these elements such as citrate in order to limit the particle size. In a particular embodiment, in combination or independently of the above, the aqueous solution containing an oxo-hydroxo salt of the element M is in the form of a sodium, potassium or ammonium salt. The pH of the aqueous solution containing an oxo-hydroxo salt of the element M is adjusted such that the precipitation reaction results in the synthesis of the portion with formula XₐL_{b}(MₚO_{q}) [for particles with formula XₐL_{b}(MₚO_{q})]. The oxidation states of the elements X, L and M are those of these elements in the final particle.

In a particular embodiment, the aqueous solution containing the elements X′ and A (or containing the element A) is in the form of chlorides, nitrates or acetates. In a particular embodiment, the aqueous solution containing the elements A and X′ (or containing the element A) may also contain complexing agents for these elements, such as citrate, in order to limit the particle size. In a particular embodiment, in combination or independently of the above, the aqueous solution containing an oxo-hydroxo salt of the element M′ is in the form of a sodium, potassium or ammonium salt. The pH of the aqueous solution containing an oxo-hydroxo salt of the element M′ is adjusted such that the precipitation reaction results in coating the portion with formula XₐL_{b}(MₚO_{q}) with a portion with formula AₑX′_{f}(M′_{p′}O_{q′}) or with a portion with formula Aₑ(M′_{p′}O_{q′}). The oxidation states of the elements A, X′ and M′ will be those of these elements in the final particle.

Step (2) is carried out in the presence of portions with formula XₐL_{b}(MₚO_{q}) synthesized in (1), i.e. step (2) is in particular carried out in the dispersion of the portions with formula XₐL_{b}(MₚO_{q}) as obtained at the end of step (1), or after the dispersion of the portions with formula XₐL_{b}(MₚO_{q}) as obtained at the end of step (1) has been purified to eliminate the salts of counter-ions.

In one embodiment, the aqueous solution containing the elements X′ and A (or containing the element A) and the aqueous solution containing the element M′ are successively added to the dispersion of the portions with formula XₐL_{b}(MₚO_{q}) as obtained at the end of step (1), the second solution being added slowly dropwise. In another embodiment, the aqueous solution containing the elements X′ and A (or containing the element A) and the aqueous solution containing the element M′ are added simultaneously to the dispersion of the portions with formula XₐL_{b}(MₚO_{q}) as obtained at the end of step (1), each of the two solutions being added slowly dropwise. The mode of adding the two solutions to the dispersion of the portions with formula XₐL_{b}(MₚO_{q}) as obtained at the end of step (1) and their concentration are controlled such that coating the portions with formula XₐL_{b}(MₚO_{q}) occurs in a manner which is preferential over separate precipitation of the portions with formula AₑX′_{f}(M′_{p′}O_{q′}) or portions with formula Aₑ(M′_{p′}O_{q′}). The skilled person could modify the modes of addition described above or vary the dilution of the added solutions.

In a particular embodiment, the coprecipitation reaction for synthesizing the portions with formula XₐL_{b}(MₚO_{q}) and the coprecipitation reaction for coating the portions with formula XₐL_{b}(MₚO_{q}) with the portion with formula AₑX′_{f}(M′_{p′}O_{q′}) or with formula Aₑ(M′_{p′}O_{q′}) are carried out in succession directly and without interruption.

In a particular embodiment, when M and M′ are identical, the dispersion of the portions with formula XₐL_{b}(MₚO_{q}) obtained directly from their synthesis may contain a quantity of M (or M′) ions sufficient to coat, the portions with formula XₐL_{b}(MₚO_{q}) with the portion with formula AₑX′_{f}(M′_{p′}O_{q′}) or the portion with formula AₑX′_{f}(M′_{p′}O_{q′}), such that only one aqueous solution containing the elements X′ and A (or containing the element A) is added in step (2).

In a particular embodiment, step (3) comprises or consists of purification of the particles in order to eliminate the counter-ion salts.

In a particular embodiment, the method comprises a final step consisting of sorting the particles according to their size, by centrifugation.

The application also proposes particles having the above definition, in particular particles with formula XₐL_{b}(MₚO_{q})/AₑX′_{f}(M′_{p′}O_{q′}) or with formula XₐL_{b}(MₚO_{q})/Aₑ(M′_{p′}O_{q′}), obtained by the method described above.

The application also concerns a pharmaceutical composition comprising particles as defined in the present application or a composition as defined in the present application, and a pharmaceutically and/or physiologically acceptable vehicle. The term “pharmaceutical composition” means a composition intended for diagnostic use and/or therapeutic use, not only in humans but also in animals, in particular in mammals and/or pets (veterinary use). The term “pharmaceutically and/or physiologically acceptable vehicle” means an agent which is suitable for using the pharmaceutical composition in contact with a living being (for example a non-human mammal, and preferably a human being) and is thus preferably non-toxic, like the excipients. Examples of such physiologically and/or pharmaceutically acceptable vehicles are water, a saline solution, in particular a physiological solution, solvents which are miscible in water, sugars, binders, pigments, vegetable or mineral oils, polymers which are soluble in water, surfactants, thickening or gelling agents, preservatives, and alkalinizing or acidifying agents. Excipients which may be contained in the pharmaceutical composition of the invention include sugars such as lactose, sucrose, mannitol, or sorbitol, preparations based on cellulose, for example corn, wheat, rice or potato starch, gelatine, gum, gum tragacanth, methyl cellulose, hydroxypropylmethyl cellulose, sodium carbomethylcellulose and physiologically acceptable polymers such as polyvinylpyrrolidone (PVP). In a particular embodiment, the excipients or vehicles are intended for the preparation of a pharmaceutical composition in accordance with the invention as an injectable solution, in particular as a solution which can be injected intravenously.

In a particular embodiment, the pharmaceutical composition comprises, as an active substance, in the range 0.1 to of particles in accordance with the invention, in particular in the range 0.1 to 0.6 g/mL or in the range 0.2 to 0.5 g/mL.

In a particular embodiment, preferably in combination with the embodiment in the preceding paragraph, the pharmaceutical composition in accordance with the invention is formulated as an injectable solution, in particular in the form of a solution which is intravenously injectable (IV), and in particular in the form of pre-filled bottles or syringes.

The application also proposes the use of particles, compositions or pharmaceutical compositions in accordance with the invention in imaging, in particular medical imaging, in particular in diagnostic imaging. The particles, compositions or pharmaceutical compositions of the invention may be used in vitro, in particular on a cell culture or on an organ which has been previously removed ex vivo, or preferably in vivo. Use in vivo includes the use in an animal, in particular in mammals, in particular in pets (veterinary use) or in humans (patients).

Thus, the application proposes the use of particles, compositions or pharmaceutical compositions in accordance with the invention in imaging, in particular on laboratory animals (mice, rats, primates etc.), in particular for research or investigative purposes, or for the purposes of the development of molecules for diagnostic and/or therapeutic ends.

The application also proposes the use of particles, compositions or pharmaceutical compositions in accordance with the invention as diagnostic agents in a patient or an animal, preferably a mammal (diagnostic use). In a particular embodiment, the particles, compositions or pharmaceutical compositions of the invention are used for exclusively diagnostic purposes, excluding their use for therapeutic purposes.

In one embodiment, the application proposes the use, in particular in vivo, of particles, compositions or pharmaceutical compositions in accordance with the invention in implementing at least one imaging technique (in particular one or a combination of two or three techniques) selected from the group consisting of MRI, optical imaging, optical oxidant detection, positron emission tomography (PET), tornodensitometry (TDM) and ultrasound imaging (for example ultrasound scan). The expression “combination” or “in combination” means that the imaging technique(s) is (are) carried out on the same subject (patient or animal) during the same investigation session, in particular diagnostic investigation, i.e. signals (in particular images) from the imaging technique(s) are acquired following a single injection of particles, compositions or pharmaceutical compositions of the invention or at most following two injections of the same particles, the same compositions or the same pharmaceutical compositions of the invention [if signal acquisition has to take longer than the clearance period for the particles in the subject (patient or animal, preferably mammal, under investigation]. Thus, the acquisition of signals by the various imaging techniques respectively carried out may be slightly spread out over time as long as the imaging techniques are deployed during the same investigation session, in particular a diagnostic investigation. The combination of the various imaging techniques using the particles, compositions or pharmaceutical compositions of the invention allows for co-localization of signals or images respectively acquired by these multiple techniques.

In one embodiment, the application proposes the use of particles, compositions or pharmaceutical compositions of the invention in MRI (or for diagnosis by MRI, or for diagnosis employing the MRI technique).

The application also proposes the use of particles, compositions or pharmaceutical compositions of the invention as multimodal agents (in particular bimodal or trimodal) in diagnostics using at least two imaging techniques selected from the group consisting of MRI, optical imaging, optical oxidant detection, positron emission tomography, tomodensitometry and ultrasound imaging. In another embodiment, the application proposes the use of particles, compositions or pharmaceutical compositions in accordance with the invention as multimodal agents (in particular bimodal or trimodal) in imaging, in particular in MRI in combination with at least one, in particular one, imaging technique selected from the group consisting of optical imaging, optical oxidant detection, positron emission tomography, tomodensitometry and ultrasound imaging.

The expression “in MRI in combination with at least one, in particular one, imaging technique selected from the group consisting of optical imaging, optical oxidant detection, positron emission tomography, tomodensitometry and ultrasound imaging” encompasses the use of particles, compositions or pharmaceutical compositions in accordance with the invention, in MRI in combination with optical imaging, in MRI in combination with optical oxidant detection, in MRI in combination with positron emission tomography, in MRI in combination with tomodensitometry or in MRI in combination with ultrasound imaging (bimodal imaging).

In one embodiment, the particles, compositions or pharmaceutical compositions in accordance with the invention are used in MRI in combination with optical imaging. Advantageously, the use of a particle in accordance with the invention (having MRI contrast agent properties and luminescent properties) can reduce the scan times by improving the contrast and simultaneously allowing for rapid optical imaging by combining the complementary advantages of optical techniques in terms of speed of acquisition and sensitivity at low concentrations with deep penetration of tissues with MRI.

In one embodiment, the particles, compositions or pharmaceutical compositions in accordance with the invention are used in MRI in combination with the optical detection of oxidants. Advantageously, the use of a particle in accordance with the invention (having MRI contrast agent properties and oxidant detection properties) means that tissues can be imaged by MRI and the production of oxidants linked, for example, to an inflammation site can be detected by injecting a single product. In this embodiment, the luminescent ions of the particle must already be in a valency state such that they are capable of undergoing oxidation.

In one embodiment, the particles, compositions or pharmaceutical compositions in accordance with the invention are used in MRI in combination with positron emission tomography. The emission of positrons by radio-isotopes appropriate to the emission of positrons, such as ¹¹C, ¹³N, ¹⁵O, ¹⁸F, ⁶⁴Cu, ⁸⁶Y or 124I is followed by a reaction with electrons and emission of γ photons the penetration depth of which is unlimited at the scale of biological samples, which means that PET is the imaging technique with the highest sensitivity, allowing the determination of the local concentration of the radio-isotope and detection of a single abnormal cell (Hahn et al, 2011). Thus, PET is appropriate for detecting the appearance of a cancer before any macroscopic changes can be seen. Advantageously, the use of a particle in accordance with the invention (having MRI contrast agent properties and carrying a radio-isotope) means that the high sensitivity of PET can be combined with localization of the PET signal in the body of the animal or patient examined by MRI.

In one embodiment, the particles, compositions or pharmaceutical compositions in accordance with the invention are used in MRI in combination with tomodensitometry. The contrast generated by the TDM is essentially between the bones and other parts of the body. Thus, TDM can provide complementary information to MRI where the contrast is generated between regions containing water, namely between different types of tissues. In addition, TDM can provide three-dimensional images with a resolution comparable to that of MRI (Frullano and Meade, 2007). In the particular case in which A, the paramagnetic lanthanide ion, is Gd, the high electronic density of the gadolinium atom makes the particles of the invention an appropriate contrast agent for TDM in combination with MRI.

In one embodiment, the particles, compositions or pharmaceutical compositions in accordance with the invention are used in MRI in combination with ultrasound imaging. In a particular embodiment and in the particular case of this combined use, the particles of the invention are contained in large numbers in polymeric microspheres or microbeads prepared prior to administering them to the subject of the investigation (Hahn et al. 2011).

The expression “in MRI in combination with at least one, in particular one, imaging technique selected from the group consisting of optical imaging, optical oxidant detection, positron emission tomography, tomodensitometry and ultrasound imaging” encompasses the use of particles, compositions or pharmaceutical compositions in accordance with the invention, in MRI in combination with two imaging techniques selected from optical imaging, optical oxidant detection, positron emission tomography, tomodensitometry and ultrasound imaging (trimodal imaging). In a particular embodiment, the particles, compositions or pharmaceutical compositions in accordance with the invention are used in MRI in combination with optical oxidant detection and optical imaging, in MRI in combination with optical oxidant detection and positron emission tomography, in MRI in combination with optical oxidant detection and tomodensitometry or in MRI in combination with optical oxidant detection and ultrasound imaging.

The invention also proposes:
the use of particles, compositions or pharmaceutical compositions for the preparation or the manufacture of a diagnostic composition, i.e. intended for use in one or a combination of several, in particular 2 or 3, imaging technique(s) as defined above; and
particles, compositions or pharmaceutical compositions for their use in imaging, in particular as diagnostic agents, as multimodal diagnostic agents (in particular bimodal or trimodal) or in diagnostics employing one or a combination of several, in particular 2 or 3, imaging technique(s) as defined above.

The invention also proposes a method for the acquisition of a signal, in particular image(s), by MRI, optical imaging, optical oxidant detection, PET, TDM or ultrasound imaging, or by a combination of at least two (in particular two or three) of these techniques as defined above, in an animal, in particular a mammal, or in a patient, employing particles, compositions or pharmaceutical compositions in accordance with the invention comprising:

a) excitation of the particles or the medium containing the particles; and

b) acquisition of at least one signal (in particular an image) associated with said particles after the excitation.

The invention also proposes a method for the acquisition of a signal, in particular image(s), by MRI, optical imaging, optical oxidant detection, PET, TDM or ultrasound imaging, or by a combination of at least two (in particular two or three) of these techniques as defined above, in an animal, in particular a mammal, or in a patient, employing particles, compositions or pharmaceutical compositions in accordance with the invention comprising:

a) administration, in particular intravenously, of particles, compositions or pharmaceutical compositions in accordance with the invention, to the animal or to the patient;

b) excitation of the particles or the medium containing the particles; and

c) acquisition of at least one signal (in particular an image) associated with said particles after the excitation.

The term “excitation”, means the application of a magnetic field (MRI), scanning with a beam of X rays (TDM), with light at a particular wavelength (optical imaging) and/or ultrasound (ultrasound imaging) of the subject (animal or patient), as a function of the imaging technique or techniques employed in the diagnostics.

The term “medium containing the particles”, means the biological liquid or the tissue into which the particles of the invention have been administered, or the biological liquid or the tissue in which the particles of the invention are localized or become concentrated (in particular due to targeting) following their administration.

The diagnostic applications of the particles, compositions or pharmaceutical compositions in accordance with the invention are numerous and correspond to the conventional applications of MRI, optical imaging, optical oxidant detection, TDM, PET or ultrasound imaging techniques. By way of example, the particles, compositions or pharmaceutical compositions are used in implementing the imaging techniques as defined above, for the diagnosis of many disorders, particular non-limiting examples of which are disorders linked to the brain, the spinal cord, the large vessels, arteries, intrathoracic organs (for example the heart), spine, digestive and pelvic viscera, muscles, joints and adjacent structures, tendons, ligaments and peripheral nerves and tumour cells. In particular, the particles, compositions or pharmaceutical compositions are used, in a non-limiting manner and as a function of the imaging technique or combination of imaging techniques employed, for the diagnosis of coronary disease, valve disease, cardiomyopathies, congenital cardiopathies, pericardial disease, congenital heart defects, tumours (bone, cardiac, lymphomas, pulmonary nodules, upper aerodigestive pathways, liver localization of digestive cancers, melanomas, breast cancers, gynaecological cancers), inflammatory neurological diseases, slipped discs, discosomatic diseases, traumatic lesions of the spine and cord, infectious spondylodiscitis, arterio-venous defects and degenerative cerebral diseases such as Alzheimer's and Parkinson's.

The application also proposes the use of particles, compositions or pharmaceutical compositions in accordance with the invention in imaging, in particular in medical imaging, in particular in diagnostics or in diagnostics imaging, as defined above, and simultaneously as a drug or for therapy. The term “simultaneous” or “simultaneously”, means that the acquisition of signals (in particular images) from the imaging technique(s) and the therapeutic step for treatment of the subject (animal or patient) are carried out, in the same subject, during the same investigative session, i.e. following a single injection of the particles, compositions or pharmaceutical compositions in accordance with the invention.

In addition to the application in imaging or the diagnostic application described in detail above, the particles of the invention may also be used as a drug or for therapy, the active ingredient possibly being the particle itself or a therapeutic molecule bound to the particle.

In one embodiment, the particle of the invention, in its uncoated form, itself constitutes the active ingredient of the drug, at least in part. When in the definition of the particle, A is Gd, neutron capture therapy (NCT) may be carried out, which relies on the huge absorption section for neutrons possessed by Gd, in particular its ¹⁵⁷Gd isotope. Thus, when neutrons (n) are captured, the ¹⁵⁷Gd nucleus undergoes a nuclear reaction:

¹⁵⁷Gd+n→¹⁵⁸Gd*→¹⁵⁸Gd+γze⁻, which causes the prompt emission of a high energy γ ray, with an energy which can reach 7.8 MeV, along with several electrons, in particular z electrons, essentially of the Auger type deriving from internal conversion with energies≤41 keV (De Stasio et al, 2001). The Auger electrons are highly ionizing over short distances of the order of tens of nanometres. These electrons may cause the rupture of double stranded DNA in tumour cells and lead to necrosis. Thus, it is advantageously possible to couple, via a particle of the invention in which A is Gd, imaging by MRI and NCT (see Bridot et al. (2009) on particles with a core of Gd₂O₃). NCT is appropriate for the treatment of cerebral tumours, in particular glioblastoma multiforme. In one particular embodiment, the particles, compositions or pharmaceutical compositions in accordance with the invention are used as a diagnostic agent or as a contrast agent in MRI, and simultaneously in the treatment of cerebral tumours.

In another embodiment, the particle of the invention in its coated form constitutes the active principle of a drug, the particle being used in particular as a drug transport vehicle. Reference in this case should be made to particles in accordance with the invention which carry therapeutic molecules (for example anticancer molecules) and, optionally, targeting molecules and/or stealth agents. Using the particles of the invention simultaneously as a diagnostic agent, in particular an MRI contrast agent, and as a vehicle for transporting therapeutic molecules has the advantage of allowing the progress and/or accumulation of the drug at the target site to be monitored by MRI, thus allowing the doses and the administration intervals to be adjusted for a maximum effect. This use is particularly appropriate for the treatment of disorders which can be diagnosed using the imaging techniques mentioned above, in particular MRI.

In a particular embodiment, the particles, compositions or pharmaceutical compositions in accordance with the invention are used as a diagnostic agent or as a contrast agent in MRI and in the treatment of tumours.

The invention also proposes:
particles, compositions or pharmaceutical compositions in accordance with the invention for their simultaneous use in imaging, in particular as a diagnostic agent or as an MRI contrast agent, and as a drug;
particles, compositions or pharmaceutical compositions in accordance with the invention for their simultaneous use in imaging, in particular as a diagnostic agent or as an MRI contrast agent, and as a drug in the treatment of tumours; and
the use of particles, compositions or pharmaceutical compositions for the preparation or the manufacture of a pharmaceutical composition intended simultaneously for carrying out imaging techniques, in particular MRI, and for the treatment of tumours.

The invention also proposes a method for treating a subject, in particular for treating a subject with tumour(s), comprising:
administering particles, compositions or pharmaceutical positions to the subject;
excitation of the particles; and
monitoring the progress and/or accumulation of the particles, in particular in the tumour, after the acquisition of at least one signal (in particular an image) associated with said particles after excitation.

Irrespective of whether the diagnostic application is used alone or in the context of a simultaneous diagnostic and therapeutic application, the doses employed will be those routinely recommended for MRI techniques. In one embodiment, the dose administered to the subject is from 0.01 to 0.5 mmol/kg, in particular 0.05 to 0.3 or 0.01 to 0.2 mmol/kg (in mmol of the paramagnetic ion or ions).

The term “comprising”, with which “including” or “containing” is synonymous, is an open term and does not exclude the presence of one or more additional element(s) or ingredient(s) or additional method step(s) which are not explicitly indicated, while the term “consisting” is a closed term which excludes the presence of any other additional element or ingredient or additional step which is not explicitly disclosed.

To facilitate reading of the present application, the description has been divided up into various paragraphs and sections. It should not be assumed that these divisions disconnect, the substance of one paragraph or section from that of another paragraph or section. In contrast, the present description encompasses all possible combinations of the various paragraphs, sections and sentences it contains.

The following examples are given purely by way of illustration. They do not in any way limit the invention.

### EXAMPLES

### I. Methods and Apparatus

### 1.1. Preparation of Reagents

Sodium orthovanadate Na₃VO₄ (purity 99.9%, M=183.91 g/mol, Alfa Aesar, Schiltigheim, France) was dissolved in ultrapure water with a specific resistance of at least 18 M Ohm cm to a final concentration of 0.1M. The pH was adjusted to 12.5-13.0. Rare earth nitrates were dissolved in ultrapure water to a final concentration of 0.1M. The solutions were prepared from Y(NO₃)₃6.H₂O (purity 99.8%, M=383.01 g/mol, Sigma Aldrich, St. Quentin Fallavier, France) and Gd(NO₃)₃.6H₂O (purity 99.9%. M=451.36 g/mol, Alfa Aesar) and were used as prepared. For the synthesis of particles doped with Eu, rare earth nitrate solutions were mixed by volume to the desired Eu concentration, giving a solution with a total rare earth concentration of 0.1M.

1.2. Synthesis of Y_{0.6}Eu_{0.4}VO₄ GdVO₄ Particles

Particles of the core/shell type containing a core with formula Y_{0.6}Eu_{0.4}VO₄ and a shell with formula GdVO₄ were synthesized. Particles with a dimension of approximately 40 nm (i.e. a radius of 20 nm) were obtained. The volumetric ratio between the volume of the core (V_{c}) and the volume of the shell (Vₛ) was calculated by using a value of 5 nm for the thickness of the shell. The volume of the shell is given by Vₛ=V_{NP}−V_{c}=4/3π(r_{NP}−r_{c})³ where V_{NP} is the volume of the particle. The following volumetric ratio was obtained:

Vₛ/V_{c}=(r_{NP}³−r_{c}³)/r_{c}³=(r_{NP}/r_{c})³−1=(20/15)³−1=1.37

For a total volume of 0.1 M lanthanide solutions of 75 mL, this corresponded to a 31.5 mL core and 43.5 mL shell solution of lanthanides. In view of the stoichiometry of the particle, the solution of lanthanides in the core was itself a mixture of 60% (vol/vol) of Y(NO₃)₃ solution and 40% (vol/vol) of Eu(NO₃)₃ solution. For the shell, a pure solution of Gd(NO₃)₃ was used.

The following method was carried out to synthesize the particles:

75 mL of a 0.1 M solution of sodium vanadate, pH 12.5-13.0 was placed in a 250 mL Erlenmeyer flask and stirred vigorously at ambient temperature. A mixture containing europium nitrate and yttrium nitrate was added at a flow rate of 1 mL/min using a peristaltic pump. Following addition of the solution forming the core, the solution of gadolinium nitrate forming the shell was immediately added at the same flow rate. At the end of all of these additions, the dispersion was left, with stirring, for an additional 30 minutes and then underwent the purification procedure as described in section 1.3 below.

### 1.3. Purification

The coarse dispersion of particles obtained was purified by dialysis or centrifuging in order to eliminate counter-ions in solution. A dialysis was carried out using Spectra/Por regenerated cellulose dialysis membranes (MWCO 12-14 kDa, Spectrum Labs, Rancho Dominguez, Calif., USA) against ultrapure water until the conductivity of the dispersion of particles was less than 100 μS·cm⁻¹. For large volumes, purification by centrifuging was carried out. The dispersion was centrifuged at 26323 g for 20 minutes. The supernatant was eliminated and the precipitate was redispersed in ultrapure water. The centrifuging-taking up into dispersion steps were repeated 3 to 5 times depending on the concentration factor and until the conductivity of the particles taken up into dispersion of less than 100 μS·cm⁻¹ was obtained.

### 1.4. Size Selection

The size selection was carried out using two centrifuging steps. The dispersion was firstly centrifuged at 500 g for 2 minutes and the supernatant obtained was centrifuged afresh at 1000 g for 2 minutes to eliminate the aggregates and very coarse particles. The supernatant contained a dispersion of particles with a good compromise between a small size distribution and a high yield. Characterization by a dynamic light diffusion technique (number average values) provided a hydrodynamic diameter of 55 nm with a distribution width of 16 nm.

### 1.5. Measurement of Relaxation Time

The relaxation times relating to the particles obtained were measured on a Bruker minispec NMS 120 relaxometer (Bruker, Rheinstetten, Germany) operating at a proton resonance frequency of ω/2π=20 MHz and a temperature of 37° C. In order to avoid any confusion between angular frequency ω and the frequency v, here, all of the frequencies given in Hz correspond to ω/2π=v. The spectrometer was calibrated using standard water/oil mixtures with known component ratios, in accordance with the manufacturer's instructions. The pre-diluted samples were further diluted directly in 10 mm NMR (nuclear magnetic resonance) tubes using a series containing 10×1 mL samples. All of the dilutions were made using ultrapure water. The tubes were sealed and placed in a water bath at 37° C. for at least 10 minutes before measurement, The relaxation times T₁ were determined by using the inversion-recovery pulse sequence with a repetition time TR=5 s. The pulse separation time TI was adjusted until the condition TI≈0.6 T₁ was satisfied. To measure the relaxation time T₂, the CPMG (Carr-Purcell-Meiboom-Gill) pulse sequence was employed using a repetition time TR=8 s. In general, 100 echoes with an echo time TE between 0.5 and 2 ms depending on the concentration of the sample were recorded. The TE was adjusted manually in order to record complete extinction of the magnetism during 100 echoes. In both cases, the software of the device carried out the adjustment of the measured recovery of the magnetisation and the corresponding relaxation times were displayed directly with their error bars.

### 1.6. Analysis of Relaxation Data

The relaxivities per particle were determined by firstly calculating the volume of a particle. To this end, the nanoparticles were assumed to be homogeneous in dimension and spherical with a diameter equal to the number average diameter determined by DLS (dynamic light scattering). In the case of silicate particles, the diameter of the uncoated (i.e. unmodified) particles was used. The number of Gd ions per particle was evaluated using the unit cell dimensions a=b=7.204 Å and c=6.338 Å obtained for GdVO₄ for all of the samples, a number of 4 formula units per unit cell and the stoichiometric factor corresponding to the composition of the respective particle. The relaxivity per particle was then obtained by multiplying the relaxivity per Gd ion by the number of Gd ions per particle.

### 1.7. Acquisition of Luminescence Spectra.

The dispersion of particles was pre-diluted so that it appeared almost transparent and was transferred into a 2 mm QS 100 quartz cell (Hellma, Müllheim, Germany). The emission spectra were recorded using a Hitachi F-4500 fluorescence spectrophotometer (Hitachi, High-Tech, Tokyo, Japan). Slits with a spectral width of 2.5 nm were used in the excitation and emission paths and scanning was carried out at a speed of 240 nm/min. In order to acquire the emission spectrum, a GG-375 high pass filter (Schott, Mainz, Germany) was placed in the detection path. Luminescence was excited at 280 nm and the emission was recorded at 500 to 700 nm. The sample was diluted further for the absorbance measurements for determining the quantum yield at 280 nm when the absorbance exceeded 0.3.

### 1.8. Response to Hydrogen Peroxide

In order to measure the response to hydrogen peroxide, a dense layer of particles was spin-coated by adding 100 μl of a suspension of 94 mM (concentration of VO₄³⁻ ions) of Y_{0.6}Eu_{0.4}(VO₄)/Gd(VO₄) particles to a quartz slide. The luminescence was recorded for an excitation intensity of 1.6 kW/cm² at an acquisition speed of 1 image is for 10 minutes during the photoreduction step and for an excitation intensity of 0.3 kW/cm² at an acquisition speed of 1 image/3 s for 10 minutes during recovery, respectively. The intensity of luminescence per image was evaluated in a circular region having a homogeneous particle coverage. The luminescence signals were normalized to a value of 1 for the first analysed image during each acquisition cycle. The photoreduction and recovery values are given in the form of a percentage with respect to this first image.

### 1.9. Nanoparticles

In the same manner as for the example above, La₁₋ₓEuₓPO₄/GdPO₄ nanoparticles, La₁₋ₓEuₓPO₄/GdPO₄ nanoparticles, La₁₋ₓEuₓP_{y}V_{1-y}O₄/GdPO₄ nanoparticles and Y₁₋ₓEuₓP_{y}V_{1-y}O₄/GdVO₄ nanoparticles, with x being from 10% to 75% and y being from 0.1% to 99% can be prepared, adapting, as above, the protocols for the synthesis of La₁₋ₓEuₓPO₄, GdPO₄, La₁₋ₓEuₓP_{y}V_{1-y}O₄, GdP_{y}V_{1-y}O₄, Y₁₋ₓEuₓP_{y}V_{1-y}O₄ or GdP_{y}V_{1-y}O₄ nanoparticles. (see Buissette, V et al. Journal of materials chemistry vol 16 issue 6 p. 529-539 or Buissette V. et al. Chemistry of Materials Vol 16 issue 19 p. 3767-3773).

### II. Results

### 2.1. Relaxation Time

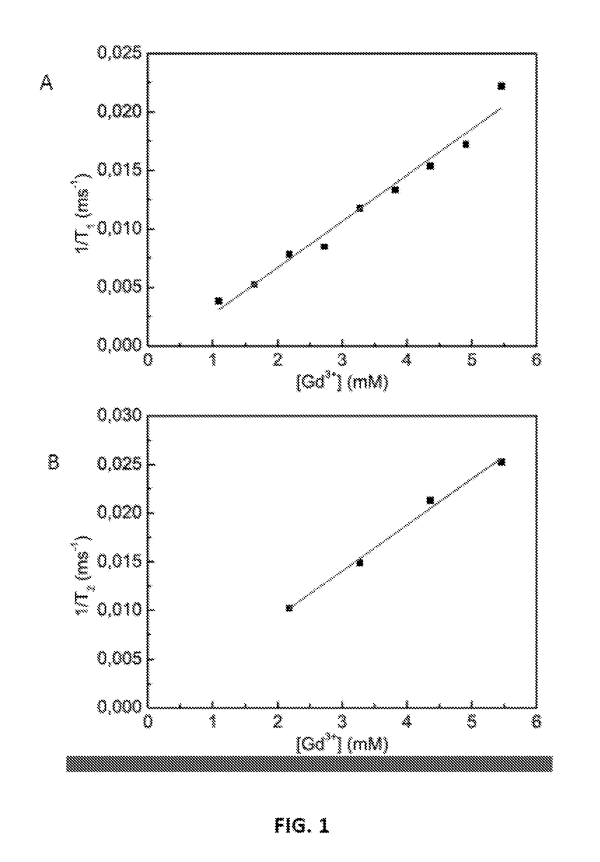

The adjustment of the relaxation times T₁ and T₂, at 20 MHz, as a function of the concentration of Gd³⁺ ions for the Y_{0.6}Eu_{0.4}VO₄ GdVO₄ particles produced the relaxivities r₁=4.0 mM⁻¹s⁻¹ and r₂=4.7 mM⁻¹s⁻¹ (FIGS. 1A and 1B).

The relaxivity of the Y_{0.6}Eu_{0.4}VO₄/GdVO₄ particles was compared with that of the other particles (see Table 2 below).

TABLE 2

| | d_{<n>} | r₁^{ion} | r₂^{ion} | r₁^{NP} | r₁^{NP} | r₂/r₁ |
|---|---|---|---|---|---|---|
| Particle | (nm)^{a} | (mM⁻¹s⁻¹) | | | | |
| GdVO₄ | 41 | 0.95 | 1.31 | 420000 | 570000 | 1.38 |
| Gd_{0.6}Eu_{0.4}VO₄ | 42 | 2.97 | 3.47 | 840000 | 980000 | 1.17 |
| Gd_{0.6}Eu_{0.4}VO₄/SiO₂ | 57 | 2.52 | 3.03 | 710000 | 860000 | 1.21 |
| DotaremTM | CC | 3.59 | 4.44 | — | — | 1.24 |
| GdCl₃ | Ion | 10.4 | 12.1 | — | — | 1.16 |
| Y_{0.6}Eu_{0.4}VO₄/ | 55 | 4.0 | 4.7 | 2.5 · 10⁶ | 2.9 · 10⁶ | 1.2 |
| GdVO₄ | | | | | | |

^{a}Number average diameter obtained by dynamic light scattering (DLS).
CC means a coordination complex.

Y_{0.6}Eu_{0.4}VO₄/GdVO₄ particles (in a core/shell organisation) are more effective for inducing the relaxation of protons (r₁^{ion} and r₂^{ion} greater than or equal to 4) than the homogeneous GdVO₄ particles and the homogeneous Gd_{0.6}Eu_{0.4}VO₄ particles. These results are attributed to the fact that more magnetically active Gd ions are located close to the surface in the Y_{0.6}Eu_{0.3}VO₄/GdVO₄ particles and can thus interact more effectively with the protons of the water compared with the Gd ions in the homogeneous particles (GdVO₄ and Gd_{0.6}Eu_{0.4}VO₄), where a portion of the Gd ions are located in the interior of the particle. These latter do not have direct contact with the water.

Furthermore, the relaxivity ratio r₂/r₁ observed with the Y_{0.6}Eu_{0.4}VO₄/GdVO₄ particles is of the same order of magnitude as that obtained with Dotarem™ and the free Gd³⁺ ions (i.e. about 1.2), only the particles constituted by pure, homogeneous GdVO₄ having a higher ratio.

### 2.2. Luminescence

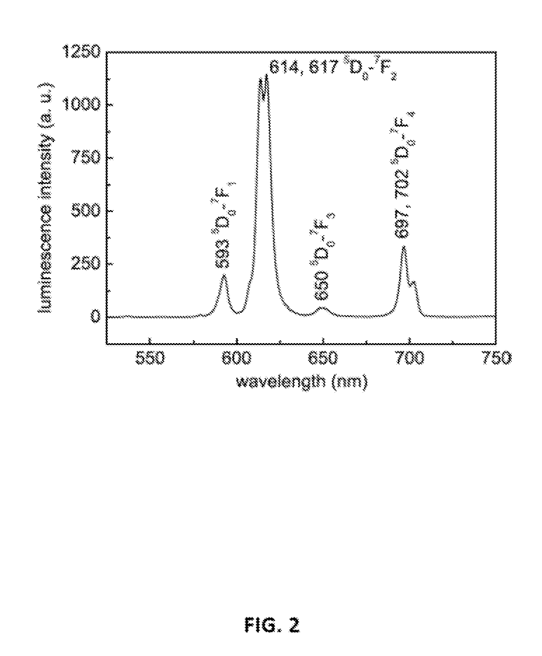

The luminescence spectrum of a suspension of Y_{0.6}Eu_{0.4}VO₄/GdVO₄ nanoparticles (in a core/shell organisation) is shown in FIG. 2. This spectrum shows a peak at 593 nm linked to the transition ⁵D₀→⁷F₁, a principal strong double peak at 616 nm (⁵D₀→⁷F₂), a very weak peak at 650 nm (⁵D₀→⁷F₃), and another double peak at 699 nm (⁵D₀→⁷F₁). This spectrum corresponds to spectra measured for YVO₄ doped with Eu in the literature (Huignard et al.; 2000). The protection of the 4f electrons of the Eu³⁺ ion by its outer electrons of the 5 s and 5 p layers results in narrow emission lines. Thus these results confirm that the two-part structure of the Y_{0.6}Eu_{0.4}VO₄/GdVO₄ particles (in particular in a core/shell organisation) does not perturb the luminescence emission spectrum of Y_{0.6}Eu_{0.4}VO₄.

### 2.3. Luminescence Quantum Yield

A calibration curve for the determination of the quantum yield was obtained from a rhodamine 6G organic fluorophore. The relative error in the adjustment was 2%. The absorption of the dispersion of nanoparticles at 280 nm was obtained as a peak on a background resulting from diffusion of incident light by the particles. The measurement of the absorbance value at 280 nm, A₂₈₀, was not very precise due to the contribution from the diffusion. A total error in the determination of the quantum yield of the order of 5% thus appears to be reasonable.

The luminescence quantum yield (Q) for the synthesis of several particles containing europium ions was thus determined. The results are summarized in Table 3.

TABLE 3
Comparison of quantum yields for the particles
Y_{0.6}Eu_{0.4}VO₄, Gd_{0.6}Eu_{0.4}VO₄ and Y_{0.6}Eu_{0.4}VO₄/GdVO₄

| Particle | Q (%) |
|---|---|
| Y_{0.6}Eu_{0.4}VO₄ | 12.4 |
| Gd_{0.6}Eu_{0.4}VO₄ | 3.8 |
| Y_{0.6}Eu_{0.4}VO₄/GdVO₄ | 10.3 |

The comparison of the luminescence quantum yield of these various particles allowed the following conclusions to be drawn:

(1) the Q value for particles with a matrix of GdVO₄ doped with europium (Gd_{0.6}Eu_{0.4}VO₄) is reduced compared with particles with a matrix of YVO₄ doped with europium (Y_{0.6}Eu_{0.4}VO₄). The GdVO₄ matrix thus appears to be less effective than the YVO₄ as regards the emission of europium ions.

(2) the Y_{0.6}Eu_{0.4}VO₄/GdVO₄ particles having two portions (a luminescent portion and a contrast agent portion) exhibited a quantum yield (Q) comparable to that of the particles constituted purely by a luminescent portion (Y_{0.6}Eu_{0.4}VO₄). This last observation demonstrates that the quantum yield is essentially determined by the direct environment of the europium ions and is only very slightly influenced by the presence of another portion, in particular a portion having a contrast agent activity. This is true even in the context of particles of the invention organized into the core/shell form, in which the luminescent portion is in the core, entirely coated by the portion having a contrast agent activity. Indeed, in view of the results shown in Table 3, neither the existence of this shell, nor its composition, is of a nature to significantly (i.e. beyond the error range) alter the quantum yield (and thus the luminescent activity) of the portion located in the core.

### 2.4 A Detection of Hydrogen Peroxide

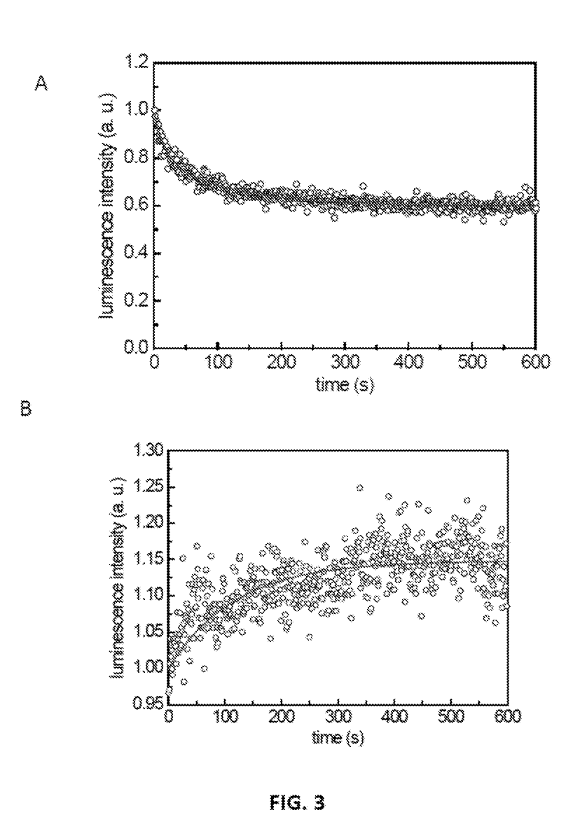

Y_{0.6}Eu_{0.4}VO_{0.4}/GdVO₄ particles were spin-coated onto a quartz slide and excited at a high laser intensity. The luminescence intensity as a function of the corresponding time is shown in FIG. 3A. The observed decrease in the intensity of luminescence confirms that photoreduction of the Eu³⁺ ions takes place in the Y_{0.6}Eu_{0.4}VO₄/GdVO₄ particles.

An adjustment in the decrease in the luminescence with a biexponential decay function was used. The decay times τ₁=17 s and τ₂=116 s as well as a reduction in intensity due to photoreduction of 40% (remaining intensity=60%), were obtained. These values are comparable with those obtained previously for Y_{0.6}Eu_{0.4}VO₄ samples (Casanova et al, 2009).

The recovery in luminescence compared with the initial intensity after photoreduction following the addition of 100 μM of H₂O₂ was 15%. The maximum recovery was reached after approximately 2 minutes, with an exponential recovery constant of τ*=119 s (FIG. 3B). These results demonstrate that these particles are capable of detecting a concentration of H₂O₂ which is as low as 100 μM.

Y_{0.6}Eu_{0.4}VO₄/GdVO₄ particles, with their core-shell organisation, constitute a powerful agent, in particular for the purposes of rnultirnodal imaging. They can be used both as a luminescent marker, as an oxidant sensor and as a contrast agent for MRI. They combine a high luminescence quantum yield. in particular necessary for the high sensitivity detection of hydrogen peroxide, with an MRI contrast which is better than that obtained with usual contrast agents.

### BIBLIOGRAPHY

Abragam, A. (1983). Principles of Nuclear Magnetism. International Series of Monographs on Physics. Oxford University Press, Oxford, UK.
Aime, S., Botta, M., Fasano, M., and Terreno, E. (1999). Acc. Chem. Res., 32(11):941-949.
Aime, S., Botta, M., and Terreno, E. (2005). Adv. Inorg. Chem., 57:173-237.
Ale A.; Schulz R.; Sarantopoulos A.; Ntziachrstos, V. (2010) Med. Phys. 37(5):1976-1986
Banci, L., Bertini, I., and Luchinat, C. (1991). Nuclear and Electron Relaxation: The Magnetic Nucleus-Unpaired Electron Coupling in Solution. Wiley VCH, Weinheim, Germany.
Bertini, I. and Luchinat, C. (1996). Proceedings of the 30th Florence NMR school. Coord. Chem. Rev., 150:1-292.
Bottrill, M., Kwok, L., and Long, N. (2006). Chem. Soc. Rev., 35(6):557-571.
Bridot, J.-L.; Dayde, D. Rivière, C.; Mandon, C.; Biliotey, C. Lerondel, S.; Sabattier, R.; Carton, G.; The Pape, A.; Blondiaux, G.; Janier, M.; Perriat, P.; Roux, S.; Tillement, O (2009) J. Mater. Chem., 19(16):2328-2335.
Bridot, J.-L.; Faure, A.-C.; Laurent, S.; Rivière, C.; Billotey, C.; Hiba, B.; Janier, M.; Josserand, V.; Coll, J.-L.; Vander Elst, L.; Muller, R.; Roux, S.; Perriat, P.; Tillement, O. (2007) J. Am. Chem. Soc., 129(16):5076-5084.
Butte, J. and Kraitchman, D. (2004). NMR Biomed., 17(7):484-499.
Caravan, P., Ellison, J., McMurry, T., and Lauffer. R. (1999). Chem. Rev., 99(9)2293-2352.
Casanova, D.; Giaume, D.; Moreau, M.; Martin, J.-L.; Gacoin, T.; Boilot, J.-P.; Alexandrou, A. (2007) J. Am. Chem. Soc. 129(42):12592-15593
De Stasio, G.; Casalbore, P.; Pallini, R.; Gilbert, B.; Sanità, F.; Ciotti, M.; Rosi, G.; Festinesi, A.; Larocca, L.; Rinelli, A.; Perret, D.; Mogk, D.; Perfetti, P.; Mehta, M.; Mercanti, D. (2001). Cancer Res., 61(10):4272-4277.
Frullano, L. and Meade, T. (2007). J. Biol. Inorg. Chem., 12(7):939-949.
Hahn, M., Singh, A., Sharma, P., Brown, S., and Moudgil, B. (2011). Anal. Bioanal. Chem., 399(1):3-27
Hifumi, H., Yamaoka, S., Tanimoto, A., Citterio, D., and Suzuki, K. (2006). J. Am. Chem. Soc., 128(47):15090-15091.
Huignard, A.; Gacoin, T.; Boilot, J.-P. (2000) Chem. Mater., 12(4)1090-1094
Huignard, A.; Buissette, V.; Laurent, G.; Gacoin, T.; Boilot, J.-P. (2002) Chem. Mater. 14(5): 2264-2269
Kim, J., Piao, Y., and Hyeon, T. (2009) Chem. Soc. Rev., 38 (2):372-390.
Kowalewski, Nordenskiöld, L., Benetis, N., and Westlund, P.-O. (1985). Prog. Nucl. Magn. Reson. Spectrosc., 17:141-185.
Lauffer, R. (1987). Chem. Rev., 87(5):901-927.
Levitt, M. (2008). Spin dynamics: Basics of nuclear magnetic resonance. John Wiley, Chichester, UK, 2nd edition.
Mastanduno, M., Davis, S., Jiang, S., diFlorio-Alexander, R., Pogue, B. and Paulsen, K. (2011) SPIE Proceedings, Molecular Imaging III, 8089:80890A
Na, H., Song, I., and Hyeon, T. (2009). Adv. Mater., 21(21):2133-2148.
Park, J.; Back, M.; Choi, E.; Woo, S.; Kim, J.; Kim, T.; Jung, J.; Chae, K.; Chang, Y.; Lee, G. (2009). CAS Nano, 3(11):3663-3669.
Schoeffel et al. SPIE/OSA European Conferences on Biomedical Optics, Munich, Germany 22-26 May 2011
Vander Elst, L., Maton, F., Laurent, S., Seghi, F., Chapelle, F., and Muller, R. (1997) Magn. Reson. Med., 38(4):604-614.

1. A method for acquiring images by MRI, optical imaging, optical oxidant detection, PET, TDM or ultrasound imaging, or by a combination of at least two of these techniques, from within a patient or an animal to which a multiplicity of luminescent and paramagnetic particles has been administered, the method comprising excitation of the administered particles and acquisition of at least one signal associated with said particles following excitation, wherein each particle comprises at least two portions, a first portion with a formula XₐL_{b}(MₚO_{q}), in which:
M is at least one element which is capable of associating with oxygen (O) to form an anion;
L corresponds to one or more luminescent lanthanide ions;
X corresponds to one or more ions which are neutral in terms of luminescence; and
p, q, a, and b are values such that the electroneutrality of XₐL_{b}(MₚO_{q}) is respected and the fraction of luminescent element, defined by a ratio of b/(b+a), is greater than 10% and less than or equal to 75%; and
a second portion with a formula AₑX′_{f}(M′_{p′}O_{q′}), in which:
M′ is at least one element which is capable of associating with oxygen (O) to form an anion;
A corresponds to one or more paramagnetic lanthanide ions;
X′ is optional and, if present, corresponds to one or more ions which are neutral in terms of paramagnetic properties; and
p′, q′, e, and f are values such that the electroneutrality of AₑX′_{f}(M′_{p′}O_{q′}) is respected and the fraction of paramagnetic element, defined by a ratio of e/(e+f), being from 80% to 100%.

2. The method of claim 1, wherein X′ is not present.

3. The method of claim 1, wherein:
M and M′, independently of each other, are selected from the group consisting of P, V, W, Mo and As;
L is selected from the group consisting of Ce, Pr, Nd, Pm, Sm, Eu, Tb, Dy, Ho, Er, Tm, and Yb;
X is selected from the group consisting of lanthanides and Bi:
A is selected from the group consisting of Ce, Pr, Nd, Eu, Gd, Tb, Ho, Er, Tm, and Yb; and
X′, when present, is selected from the group consisting of lanthanides and Bi.

4. The method of claim 3, wherein:
M and M′, independently of each other, are selected from the group consisting of P, V, and a combination thereof;
L is Eu;
X is selected from the group consisting or La, Y, Gd, and Bi;
A is Gd; and
X′ when present, is selected from the group consisting of La, Y, Gd, and Bi.

5. The method of claim 1, wherein:
the ratio b/(b+a), is from 10% to 60%;
the ratio e/(e+f) is from 90% to 100%;
p and p′ are, independently of each other, 0 or 1; and
q and q′ are, independently of each other, in the range 2 to 5.

6. The method of claim 5, wherein:
the ratio b/(b+a) is from 25% to 50%;
the ratio e/(e+f) is 100%;
p and p′ are equal to 1; and,
q and q′ are equal to 4.

7. The method of claim 6, wherein:
X is Y, L is Eu, M is P, V, or both P and V, such that the first portion has the formula YₐEu_{b}(P,V)O₄ in which the ratio b/(b+a) is from 25% to 50%; and
A is Gd and M′ is P, V, or both P and V, such that the second portion has the formula Gd(P,V)O₄.

8. The method of claim 1, wherein M is V and L is Eu.

9. The method of claim 8, wherein p is 1 and q is 4.

10. The method of claim 1, wherein the first portion and the second portion are arranged in a core/shell structure such that one such portion constitutes the core of the particle and the other such portion constitutes the shell of the particle.

11. The method of claim 10, wherein the first portion constitutes the core and the second portion constitutes the shell.

12. The method of claim 11, wherein the first portion has the formula Y_{0.6}Eu_{0.4}(VO₄) and the second portion has the formula Gd(VO₄).

13. The method of claim 1 further comprising a third portion that comprises as least one layer selected from the group consisting of a preparation layer, a layer carrying functional groups, and a layer constituted by biologically active molecules, and combinations thereof.

14. The method of claim 13, wherein the biologically active molecules are selected from the group consisting of molecules with a therapeutic activity, targeting molecules, stealth agents, fluorescent molecules, and combinations thereof.

15. The method of claim 1, the particle size is in the range of 1 to 500 nm.

16. The method of claim 10, wherein the shell is paramagnetic and/or neutral in terms of luminescence.

17. The method of claim 1, wherein the particles are employed as a pharmaceutical composition further comprising a pharmaceutically and/or physiologically acceptable vehicle.

18. The method of claim 1, wherein element L is excited directly in the visible.
